(19) United States
(12) Patent Application Publication
(10) Pub. No.: US 2018/0214160 A1
(43) Pub. Date: Aug. 2, 2018

(54) PERCUTANEOUS VASCULAR INJURY TREATMENT SYSTEMS AND METHODS

(71) Applicant: Tricol Biomedical, Inc.

(72) Inventors: Matthew Hoskins, Bend, OR (US); Alysha Wold, Beaverton, OR (US); Stephanie Wiegman, Portland, OR (US); Bill Shields, Portland, OR (US); Simona Buergi, Portland, OR (US); Simon McCarthy, Portland, OR (US); Cole Gannett, Tigard, OR (US); Ervelyn Winata, Beaverton, OR (US)

(21) Appl. No.: 15/542,626

(22) PCT Filed: Jan. 8, 2016
(86) PCT No.: PCT/US16/12732

§ 371 Date: Jul. 10, 2017

Related U.S. Application Data
(60) Provisional application No. 62/101,948, filed on Jan. 9, 2015.

(51) Int. Cl. A61B 17/132; A61B 17/135

(52) U.S. Cl. CPC A61B 17/1325; A61B 2017/12004; A61B 17/135

(57) ABSTRACT

The present invention relates to a compressive band and hemostatic dressing system and related methods of use to provide rapid, non-occlusive bleeding control, arterial injury treatment, and injury protection comfortably and reliably after vascular access procedures. The present invention may comprise some or all of the following elements: a positioner assembly, a compressive cushion component, a compression plate, a strap with a flexible hinge assembly, and a hemostatic dressing. In a preferred embodiment, the systems and methods are applied to treat and ameliorate vascular injury in a transradial percutaneous access procedure and protect against underlying vascular injury; however, the systems and methods described herein may be applied to any vascular access procedure including arm or leg vascular approaches such as the femoral, brachial, dorsalis pedis or tibial blood vessels in arterial or venous line or sheath removal or trocar removal, and in hemodialysis.

### RELATED APPLICATIONS

This application claims priority to U.S. provisional application Ser. No. 62/101,948, filed on Jan. 9, 2015, titled COMPRESSIVE RADIAL ACCESS BAND WITH HEMOSTATIC DRESSING. The entire contents of the above-referenced application is incorporated by reference herein and made part of this specification.

### FIELD OF THE INVENTION

The present invention relates to a compressive band and hemostatic dressing system and related methods of its use to provide rapid, non-occlusive bleeding control, arterial injury treatment, and injury protection comfortably and reliably after a percutaneous vascular access procedure. The compressive band and hemostatic dressing system may comprise all or a selection of the following elements: a positioner assembly, a compressive cushion component, a compression plate, a strap with a flexible hinge assembly, and a hemostatic dressing. In a preferred embodiment of the invention, the systems and methods are applied to treat and ameliorate vascular injury in a transradial percutaneous access procedure and to protect against underlying vascular injury. However, the systems and methods described herein may be applied to any vascular access procedure including, but not limited to, arm or leg vascular approaches such as access by the femoral, brachial, dorsalis pedis or tibial blood vessels, in arterial or venous line or sheath removal or trocar removal, and in hemodialysis.

### BACKGROUND OF THE INVENTION

Percutaneous vascular access procedures are commonly used to provide vascular access in procedures including, but not limited to, dialysis, radiographic percutaneous coronary artery diagnosis, and interventions to repair vascular aneurysm, interventions to replace or repair defective valves, interventions to treat coronary artery stenosis and interventions to treat aortic stenosis. During the procedure, an introducer sheath or trocar (placed through an incision injury in the vessel wall) maintains vascular access and hemostatic control. At the conclusion of the procedure, the introducer sheath or trocar is removed from the vessel and another hemostatic technique is then applied to control bleeding until the vessel wall injury closes. On removal of the sheath, the time to vessel injury wall closure will be dependent on trocar or introducer sheath size (vessel injury size), anti-coagulation regime and whether the vessel is arterial or venous. Introducer sheaths of 8 French and larger provide for longer vessel injury wall closure times. Anti-coagulation regimes including anti-platelet, direct factor Xa inhibitor, and direct thrombin inhibitor medications may also delay vessel wall closure. Typically, the preferred procedure in radial, or other extremity, arterial access is the temporary application of a simple pressure band to apply firm pressure immediately over the vessel, proximal to the vessel entry site, until standard procedure designates that the band and applied pressure can be removed without causing bleeding or hematoma. Standard compression band application times are between 120 to 720 minutes. Pressurized arterial compression bands worn for extended time can provide discomfort to the patient, and may lead to complications such as arterial or vein occlusion, extremity ischemia and neuropathy. Once the compression band is removed, the injury is covered and there is a period of observation before the patient is released to go home. The released patients are advised to refrain from activities that could lead to opening of the injured artery, thus causing rebleeding or hematoma. Should rebleeding or hematoma occur, the patients are advised further to apply direct pressure to the bleeding site and immediately seek emergency assistance.

Hemostatic materials fall into three groups: mucoadhesives, procoagulants and factor concentrators. Mucoadhesive hemostatic materials are materials that partially dissolve enabling wetting and subsequent adherence to tissue in the presence of blood to seal, and rapidly control bleeding from tissue surfaces independent of the normal clotting cascade. Mucoadhesive materials include polycationic and polyanionic hydrophilic polymeric materials. Polyacrylic acid (pKa of 4.5) is an example of a polyanionic mucoadhesive hemostatic material, while chitosan, at pH less than 6.5, is an example of a polycationic mucoadhesive hemostatic material. Chitosan at pH above 6.5 is not a mucoadhesive agent, however the C-2 amine of D-glucosamine may be converted into a cationic moiety by reactive addition, or by lowering the local aqueous environment pH below Chitosan's pKa of 6.5 (Roberts 1991). A convenient example of control of local environment pH is by forming chitosan into its dry, solid, acid salt, polycationic (poly-D-glucosammonium) by combination, in aqueous solution, with a stoichiometric equivalent, or less, of acid salt, and subsequently drying. The dried acid salt form of chitosan maintains pH below 6.5 in the presence of water or blood and provides for a range of useful, readily controlled, bioactive properties in terms of ability to readily adhere firmly to tissue, mucosa, and cellular surfaces containing polyanions such as neuraminic (sialic) acid and lipoteichoic acid.

Chitosan is a generic term used to describe linear polysaccharides that are composed of glucosamine and N-acetyl glucosamine residues joined by β-(1-4) glycosidic linkages (typically the number of glucosamines≥N-acetyl glucosamines) and whose composition is soluble in dilute aqueous acid (Roberts 1991). The chitosan family encompasses poly-β-(1-4)-N-acetyl-glucosamine and poly-β-(1-4)-N-glucosamine with the acetyl residue fraction and its motif decoration (either random or block) affecting chitosan chemistry. The C-2 amino group on the glucosamine ring in chitosan allows for protonation, and hence solubilization of chitosan in water (pKa≈6.5) (Roberts 1991).

### SUMMARY

The compressive band and hemostatic dressing system of the invention may comprise all or some of the following elements: a positioner assembly, a compressive cushion component, a compression plate, a strap with a flexible hinge assembly, and a hemostatic dressing. Preferred embodiments may include the compressive band and hemostatic dressing systems of the invention with or without the compressive cushion component.

In a generally preferred embodiment, the positioner assembly is flexible or semi-rigid with a fixed curve profile, and the strap with a flexible hinge assembly is connected to the positioner assembly and carries, or provides attachment for, each of the compression plate, the optional compressive cushion component, and the hemostatic dressing assembly. The flexible hinge assembly is provided or formed on, as part of, or by the strap and connects the strap to the positioner assembly, and may be reinforced with additional materials provided on the strap. The compression plate is located on or within the strap and is rigid with fixed flat or planar profile for placement immediately over or above the hemostatic dressing and injury. The optional compressive cushion component is provided in association with the compression plate and is in either direct or indirect contact with each of compression plate and the hemostatic dressing. If no compressive cushion component is provided, the hemostatic dressing is provided in either direct or indirect contact with the compression plate.

The present invention is an improved compressive band and hemostatic dressing systems and methods for providing simple to apply, fast, effective, non-occlusive bleeding control, vascular injury treatment, and injury protection comfortably and reliably with reduced incidence of arterial or vein occlusion, ischemia and neuropathy after, for example, a cutaneous or vascular access procedure. The present invention combines a unique compressive band system and hemostatic dressing system to achieve enhanced bleeding control with greater comfort due to the ability to effectively use the invention with reduced pressure. The combined unique compressive band system and hemostatic dressing system of the present invention also conveniently provides for the application of a hemostatic dressing at the time of compression which may be left in place for an extended period of time for continued wound management and protection during healing. The unique configuration and advantages of the present invention lead to faster, effective, safer, and more comfortable hemostasis at puncture injury sites relative to other such devices.

The positioner assembly is curved to facilitate proper placement, initial compression pressure, stabilize, and otherwise fit and secure the compressive band in place during wound treatment. In a preferred embodiment, the positioner element is prepared from a gamma irradiation stable, injection-molded, non-rigid material that may include, but is not limited to, a plasticized polyvinyl chloride (PVC) or an elastomeric polyurethane material. In another preferred embodiment the positioner assembly is a curved positioner element that is provided together with a strap, and each of the positioner assembly and strap includes a complementary mating surface to secure closure of the system, such as a hook and loop closure mating surface, to secure the compressive band and hemostatic dressing systems to an arm or leg.

The strap with a flexible hinge assembly is connected to the positioner assembly and carries, or provides attachment for, each of the compression plate, the optional compressive cushion component, and the hemostatic dressing assembly.

The flexible hinge assembly is located between the positioner assembly and the compression plate and facilitates an appropriate fit of the system to a subject or patient, and enables a simple initial application of pressure by a light to moderate pincer hold (applied between thumb and forefinger) from the caregiver that can be maintained in the compressive band and hemostatic dressing system during the initial application and strap securement to a patient's arm or leg. The distance between the compression plate and the flexible hinge assembly provided by the flexible hinge assembly also serves to remove or diminish interference of the positioner assembly with the operation of the hemostatic dressing and optional compressive cushion component.

The compression plate is rigid and attached to, housed within, located on, or connected to the flexible strap and, thus, is movable relative to the positioner assembly through the flexible strap hinge assembly. The compression plate provides for local application and maintenance of pressure over percutaneous and vascular injury sites.

In a particularly preferred embodiment, the compression plate is located or housed within the strap, in a pocket formed on or in the flexible strap, such that the compression plate is flexibly attached or connected to each of the positioner assembly and the strap terminus. As noted above, the flexible attachment provided by the flexible hinge assembly between the compression plate and the positioner assembly allows for proper placement and fitting and initial compression pressure over the injury during compressive band and hemostatic dressing system application.

A pocket to house, hold, or contain the compression plate may be formed on the strap with a flexible hinge assembly by addition of a pocket overlay material to the strap. In a preferred embodiment, the pocket overlay material is melt adhered to the strap on at least two sides such that the compression plate is accessible through lateral openings along the strap side edges. In a particularly preferred embodiment, the pocket overlay material additionally extends from the pocket towards the positioner assembly and its association with the strap provides additional reinforcement for the flexible hinge assembly.

The optional compressive cushion component may be located under a compression plate that is, in turn, associated with at least one of the strap with a flexible hinge assembly and the positioner assembly. When included, the compressive cushion component provides for enhanced control and more consistent, localized, uniformly-distributed compression over a hemostatic dressing centrally positioned over cutaneous and vascular injury sites after percutaneous vascular access. Ability to apply consistent, uniformly-distributed, light to moderate compression immediately over the hemostatic dressing and cutaneous and vascular injury sites without causing arterial or vein occlusion or neuropathy provides significant improvements in patient safety and comfort compared to commercially available compression band systems.

Embodiments of the compressive cushion component may include the compressive cushion component preformed of a substance or material into its final, delivered, support shape and size, or of the compressive cushion component as a receptacle, vessel, or container for addition or removal of a substance or material when needed. A preferred embodiment of the compressive cushion component is a balloon component that can be inflated or enlarged or deflated or collapsed conveniently with a fluid to increase or reduce compression when treating a patient. Another preferred embodiment provides the compressive cushion component filled with a fluid that is permanently contained within the compressive cushion component. “Fluid” is defined herein as a material that deforms under applied stress and includes, for example, gases, liquids, and plastics. The preferred balloon component embodiment provides for flexibility and ease of use in adding and removing uniformly-distributed, support compression by addition or removal of a fluid.

In a preferred embodiment, the compressive cushion component maintains consistent and reliable internal pressure with addition of a fixed amount of air into the empty balloon bladder to within ±35% over 120 minutes under ambient temperature and pressure conditions and under initial internal pressure of 7±1 pounds per square inch, or more preferably to within ±25% over 120 minutes under ambient temperature and pressure conditions and under initial internal pressure of 7±1 pounds per square inch, and most preferably to within ±20% over 120 minutes under ambient temperature and pressure conditions and under initial internal pressure of 7±1 pounds per square inch.

In a particularly preferred embodiment, the compressive cushion component does not extend under, and is not located under, the positioner assembly. The entirety of the compressive cushion component is substantially located under the compressive plate and the outer perimeter edge of the compressive cushion component located closest to the positioner assembly does not extend past a strap flexible hinge mechanism that connects the strap to the positioner assembly. When provided in this advantageous configuration, the compressive cushion is primarily subject to pressure provided by the compression cushion component and compressive plate and is not pinched or deformed due to direct pressure from the positioner assembly, and facilitates placement and removal of the compressive band system while leaving the hemostatic dressing assembly in place.

The compressive cushion component may be a singular compressive cushion without a supportive or protective frame, tray, case, or the like, or may include such features. The compressive cushion component may be asymmetric or symmetric in shape. Any supportive or protective features may be provided separately or as integrally formed parts of the compressive cushion component. Thus, a separate compressive cushion may, or may not, be provided within or together with a supportive frame, tray, case, or the like. It is also contemplated that the entire compressive cushion component may consist only of a compressive cushion.

The compressive cushion component or portions thereof may be separate from but connected to, attached, or integrally formed with, at least one of the strap and the compression plate. Whether the compressive cushion component is connected to, attached, or integrally formed with either the strap and the compression plate may depend on which component provides an exposed injury-facing surface suitable for attachment. In a particularly preferred embodiment, the compressive cushion component is attached along only one edge to the strap area located under the compression plate (which may be provided in a strap pocket configuration). In this embodiment, the compressive cushion component attaches to a region of the strap area under the compression plate that is most remote from the positioner assembly. In various embodiments, the compressive cushion component may be attached in an offset manner or centrally under the compression plate, and the attachment point may be conjoined or proximal to the flexible hinge assembly side of the compression plate or may be conjoined or proximal to the compression plate or strap area side of the compression plate opposite the flexible hinge assembly. In a preferred embodiment, the manner of connection to, attachment, or integral formation of the compressive cushion component with at least one of the strap and the compression plate is flexible to allow for changes in size to the compressive cushion component.

The compressive cushion component may be a balloon component that can be inflated or deflated conveniently with fluid as needed to achieve a desired compression over the hemostatic dressing. The balloon component may be a singular or multi-part balloon without a supportive or protective frame, tray, case, pressure indicator or the like, or may include such features. The balloon component may be asymmetric or symmetric in shape. Any such supportive or protective or pressure indicator features may be provided separately or as attached or integrally formed parts of the balloon component. In one embodiment, the entire balloon component may consist of a singular inflatable bladder. The balloon component or portions thereof, may be separate from but connected to, or integrally formed with, at least one of the compression plate and strap. A preferred embodiment of the balloon component includes a feed tube that connects the interior of a balloon with a fluid line and a pump to regulate pressure. The feed tube may include a valve. In the case the fluid is air then the feed tube is an airline.

The hemostatic dressing of the present invention is removable or detachable from its support surface. The hemostatic dressing may comprise chitosan salts, derivatized chitosan or other mucoadhesive hemostatic agents that, when wet, adhere to, seal, and protect tissue and mucosa injuries. In a preferred embodiment, the hemostatic dressing comprises mucoadhesive chitosan and is unbound, separated, or detached from its support surface upon removal of the positioner assembly, compressive cushion component (when used), compression plate, and strap from an injury site. In a preferred embodiment, the hemostatic dressing includes a tab protruding from the edge of the non-injury facing side of the dressing (hemostatic dressing assembly). The non-injury facing side of the hemostatic dressing is associated with or attaches to the compression band via the compression plate (that may or may not be housed within, or located above, a strap) or the compressive cushion component. The tab is permanently and securely attached to the non-injury facing side of the hemostatic dressing surface, and may include, but is not limited to extension of polyurethane, polyester, polyethylene, polypropylene films attached to the non-injury facing side of the dressing surface and that extend beyond one edge of the hemostatic dressing.

The tab acts as a manually accessible support feature during practice of the invention to allow, by single or double finger pressure on the tab against the patient, leaving the hemostatic dressing assembly in place against the cutaneous or percutaneous access injury, while at the same time allowing removal of the positioner assembly, compressive cushion component (when used), compression plate, and strap (which may be referred to collectively as the “band assembly”) from injury site and patient. The supported hemostatic dressing subsequently is secured in place over the injury by a standard procedure to be removed as desired. In a preferred embodiment, the hemostatic dressing is composed of a protective, antibacterial, wound healing, hemostatic, mucoadhesive, chitosan composition which is left in place for at least twelve (12) hours and then is subsequently removed by the patient by applying running water over the chitosan dressing until the mucoadhesive attachment of the chitosan to the skin is sufficiently weakened to allow removal without disruption to the healing wound or the skin surface.

The hemostatic dressing and tab assembly may be held, bound, attached, or adhered to the band assembly support surface using various methods. In a preferred embodiment, the hemostatic dressing is connected to the band assembly support surface via a pressure sensitive adhesive (PSA) film layer attached to the tab release film immediately between the contacting region between tab film and compressive band support surface. A preferred embodiment of detachment method of the tab film from the support surface without PSA film layer remaining on the tab film is achieved by use of a release surface coating on the surface of the tab film in contact with the PSA film layer connecting the tab to the support surface. Release surfaces are those in which the PSA film layer can be easily peeled away without damage to the PSA film. Release surfaces are typically achieved with thin coatings of low surface energy materials applied to a support film surface. Low surface energy materials include silicones, long alkyl chain branched polymers and fluorinated polymers. Alternate embodiments of release surface may be obtained without use of low surface energy thin coatings by use of a rough surface that reduces contact area of the PSA film layer with the release surface. Alternate embodiments for release of the hemostatic dressing assembly from the compressive band support surface may include controlled or reduced surface area of contacting adhesive such as short lines of adhesive material or individual low surface area dots of hot melt or similar moderate strength adhesive. In a preferred embodiment, the hemostatic dressing may be unbound, unattached, released, or removed from the compressive band support surface on application of load directed between normal to parallel of the adhesive surface(s). Once applied, the hemostatic dressing may be unbound, unattached, or removed from the puncture injury by wetting with water or saline.

In a preferred embodiment, the hemostatic dressing is provided immediately under a flat injury-facing portion of the compression plate (that may or may not be housed within, or located above, a strap) with one edge of the hemostatic dressing aligning in parallel with the flexible hinge mechanism located between compression plate and the positioner assembly. In the compressive band and hemostatic dressing system with compressive cushion component, the hemostatic dressing is supported on the injury-facing surface of the compressive cushion component, and the compressive cushion component is associated with, or attached to, the compression plate (that may or may not be housed within, or located above, a strap).

In a preferred embodiment, the contemplated hemostatic dressing is a compressed, freeze-phase-separated, dried, interconnected-porous, chitosan acetate sponge and includes the original and currently marketed HemCon Bandage and Patch dressings (e.g., HemCon® Bandages PRO and HemCon Patch® PRO); however, it would generally be of smaller size, for example about 2.5 cm×2.5 cm. Examples of dressings (HemCon Bandage dressings) contemplated for use include those dressings and materials described in U.S. Pat. Nos. 7,371,403, 7,482,503, 7,820,872, 8,269,058, 8,313,474, 8,668,924, and 8,741,335; U.S. patent application Ser. Nos. 11/020,365, 11/202,558, 11/485,857, 11/520,230, 11/520,357, 11/541,991, 11/541,988, 11/900,854, 12/313,530, 13/122,723, and 14/211,632; and U.S. Provisional Patent Application No. 61/935,569, the contents of each of these referenced patent applications is incorporated herein in its entirety. In a preferred embodiment, the hemostatic dressing of the present invention comprises chitosan and is a freeze-phase-separated, dried, interconnected-porous, chitosan acid dressing, including an acid salt comprising one of acetic, lactic, glycolic or citric acid.

The hemostatic dressing may deliver an active ingredient to the injury site. The compositions of the present invention may further comprise an active ingredient. The active ingredient may include, but is not limited to, calcium, albumin, fibrinogen, thrombin, factor VIIa, factor XIII, thromboxane A2, prostaglandin-2a, activated Protein C, vitronectin, chrondroitin sulfate, heparan sulfate, keratan sulfate, glucosamine, heparin, decorin, biglycan, testican, fibromodulin, lumican, versican, neurocan, aggrecan, perlecan, lysozyme, lysly oxidase, glucose oxidase, hexose oxidase, cholesterol oxidase, galactose oxidase, pyranose oxidase, choline oxidase, pyruvate oxidase, glycollate oxidase and/or aminoacid oxidase, D-glucose, hexose, cholesterol, D-galactose, pyranose, choline, pyruvate, glycollate, aminoacid, epidermal growth factor, platelet derived growth factor, Von Willebrand factor, tumor necrosis factor (TNF), TNF-alpha, transforming growth factor (TGF), TGF-alpha, TGF-beta, insulin like growth factor, fibroblast growth factor (FGF), keratinocyte growth factor, vascular endophelial growth factor (VEGF), nerve growth factor, interleukin, amphiregulin, retinoic acid, erythropoietin, mafenide acetate, silver sulfadiazine, silver nitrate, nanocrystalline silver, penicillin, ampicillin, methicillin, amoxicillin, clavamox, clavulanic acid, amoxicillin, aztreonam, imipenem, streptomycin, kanamycin, tobramycin, gentamicin, vancomycin, clindamycin, lincomycin, erythromycin, polymyxin, bacitracin, amphotericin, nystatin, rifampicin, tetracycline, doxycycline, chloramphenicol, cefuroxime, cefradine, flucloxacillin, floxacillin, dicloxacillin, potassium clavulanate, clotrimazole, cyclopiroxalomine, terbidifine, ketoconazole, paclitaxel, docetaxel, imatinib, exemestane, tamoxifen, vemurafenib, ipilimumab, dacarbazine, interleukin-2, abiraterone, doxorubicin, 5-fluorouracil, tamoxifen, octreotide, sorafenib, resveratrol, ketamine, diclofenac, ibuprofen, paracetamol, codeine, oxycodon, hydrocodone, dihydromorphine, pethidine, buprenorphine, tramadol, venlafaxine, flupirtine, carbamazepine, gabapentin, pregabalin, lidocaine, prilocaine, tetracaine, benzocaine, hydrocortisone, prednisolone, betamethasone, flurometholone, dexamethasone, quercetin, diosmin, hidrosmin, curcumin and combinations thereof.

Any of the compressive cushion component, compression plate, and the strap with a flexible hinge assembly may be transparent or semi-transparent; meanwhile, the positioner assembly and hemostatic dressing may be non-transparent or opaque either in part or in whole. It is important for the caregiver to be able to monitor hemostasis status of the patient. Unhindered visualization of the hemostatic dressing, and any bleeding immediately underneath the strap, compression plate and compressive cushion component is therefore particularly preferable. The primary visualization indicator of hemostasis with use of the compressive band and hemostatic dressing system of the invention over an injury is absence of weeping or blood flow from the hemostatic dressing edges over the same injury. In a preferred embodiment of the hemostatic dressing, the hemostatic dressing may be prepared to demonstrate partial translucence when wet with blood. In an alternate embodiment of the hemostatic dressing, the hemostatic dressing may be prepared to allow wicking of a small amount of blood to the caregiver observation side of the dressing surface. Thus appearance of blood and its location within the dressing relative to the cutaneous injury site may be used as an alternate visualization indicator of the hemostasis status when a patient is wearing the compressive band and hemostatic dressing system of the invention.

It is noted that rigid materials are typically described by their flexural moduli. Flexural modulus is a measure of resistance of a material to bending stress. A material with flexural modulus at ambient room temperature (between about 20 to 26° C./68 to 79° F.) above one (1) gigapascal (GPa) is definitely rigid whereas a material with flexural modulus at ambient room temperature (between about 20 to 26° C./68 to 79° F.) below 100 megapascal (MPa) is definitely flexible and non-rigid. In one embodiment, the plasticized polyvinyl chloride (PVC) used in non-rigid components of the present invention has flexural moduli (at ambient room temperature) in the range of about 1.5 to 50 MPa. The rigid material components of the present invention have flexural moduli (at ambient room temperature) greater than about 1 GPa and may include, but are not limited to a polycarbonate, a copolyester and a acrylonitrile butadiene styrene (ABS) material and combinations thereof.

In a preferred embodiment, the compressive band and hemostatic dressing system is used to achieve fast, non-occlusive bleeding control following introducer sheath removal in a percutaneous transradial access procedure.

In a preferred embodiment, the hemostatic dressing also acts as a bactericidal antibacterial barrier over the injury site, stops opportunity for rebleeding from the site, and promotes wound healing and wound closure for up to twenty-four (24) hours after introducer sheath removal.

In a preferred embodiment of compressive band and hemostatic dressing systems and methods for injury treatment following transradial procedure, use of the invention, in conformance with device instructions and recommendations from Society for Cardiovascular Angiography and Intervention's Transradial Working Group (Rao et al. 2013), is expected to significantly reduce incidence of radial artery occlusion, ischemia and neuropathy. The compressive band and hemostatic dressing system of the invention is applied with light to moderate pressure immediately proximal to the arterial access injury for 15 to 120 minutes (the hemostatic dressing covering both arterial and percutaneous injury sites). Preferred embodiments of the hemostatic dressing, described herein, rapidly adhere to tissue after contacting blood to seal bleeding injuries and provide injury protection. Application of the hemostatic dressing, as part of a compressive band device, provides immediate injury protection, removes the need for the manual compression interval of 5 to 30 minutes required with hemostatic dressings without a band, and provides for substantial improvement in the non-occlusive hemostatic attributes of the compressive band. After detachment from, and removal of the compressive band, the hemostatic dressing may be secured in place using a securement dressing or other device against the percutaneous injury for ongoing arterial injury treatment to protect against errhysis, rebleeding and hematoma, promote wound healing and protect the injury site. Thus, the compressive band pressure in combination with the hemostatic dressing provides for immediate, non-occlusive, arterial hemostasis over an arterial access injury site. The hemostatic dressing of the invention also protects the injury site, assists the compressive band with promotion of non-occlusive hemostasis, protects against rebleeding and hematoma on compression band removal, and promotes wound healing.

In an alternative embodiment, the positioner assembly is flexible or semi-rigid with a fixed curve profile, the compression plate is rigid with fixed flat or planar profile for placement immediately over the injury and hemostatic dressing, and separate from, but flexibly connected by way of flexible hinge connector to, at least one of the positioner assembly and the strap, the strap is flexible and connected to at least one of the flat portion of the compression plate and the positioner assembly, the compressive cushion component (if present) is provided with the compression plate.

The compressive band and hemostatic dressing system and method used to provide non-occlusive bleeding control, arterial injury treatment, and injury protection comfortably and reliably after, for example, a percutaneous vascular access procedure is preferably packaged and sealed within a dry, low moisture vapor transmissible (MVTR), heat sealable, foil or metalized paper pouch, or pre-formed foil or metalized container. In the case of compressive band and hemostatic dressing system with balloon use, a preferred embodiment in the packaging in the same pouch is a syringe used for balloon inflation that is able to connect to a valve connection to the balloon; and a disposable molded support that provides for support of both the syringe and the compression band system. In the case of compressive band and hemostatic dressing system without balloon use, a preferred embodiment in the packaging in the same pouch is a disposable molded support that provides for support of the compression band system. The package and the contents of the package are sterilized by terminal gamma irradiation sterilization to at least sterility assurance level (SAL) 10⁻⁶. The sterilized and packaged system will have a shelf life when stored at or below ambient room temperature (≤26° C./79° F.) of at least forty-eight (48) months.

The present invention may be held in place, or secured to a subject or a patient receiving treatment, using any of various mechanisms. For example, the present invention may be secured like a conventional watchband, via a ratcheting mechanism, by snaps, by hook and loop closure patches or tabs (e.g., Velcro®), etc. In a preferred embodiment, the present invention is secured to a subject or a patient receiving treatment like a watchband with hook and loop closure patches or tabs. In a preferred embodiment, the hinge mechanism between the positioner assembly and the compression plate provides for initial application pressure by light to moderate hold pressure (applied between thumb and forefinger) upon each of these components from the caregiver. This pressure is maintained in the compressive band and hemostatic dressing system on initial application and strap securement to a patient's arm or leg. The securing mechanisms may be added to, connected to, attached, or integrally formed as part of the strap and, optionally, the positioning assembly. In a preferred embodiment, the securing mechanisms are provided as mating components on each of the positioner assembly and strap. The securing mechanisms are configured to be provided to, or removed from, a subject or treated patient together with the positioner assembly, compressive cushion component (when included), compression plate, and strap from an injury site.

The present invention may be used, for example, following a percutaneous vascular access procedure including, but not limited to, a percutaneous transradial access procedure according to the following steps:
1. Using a sterile technique, open the package containing the compressive band and hemostatic dressing system and transfer said system into the sterile field.
2. After catheterization, withdraw the sheath 2 to 3 cm.
3. Place the compressive band and hemostatic dressing system around the patient's extremity (forearm in the case of a transradial procedure) with the center of the hemostatic dressing 2 to 3 mm proximal to skin entry site and the positioner assembly correctly oriented (thumb side in the case of a radial procedure).
4. Apply light to moderated pressure over hemostatic dressing and injury site using thumb and forefinger pressure. It is intended that the thumb and forefinger apply compressive closure of the compression plate and positioner assembly connected through their common flexible joint.
5. Holding the light to moderate pressure of step 4, fix the compressive band and hemostatic dressing system securely around the patients arm or leg (forearm in a transradial procedure) (device will be positioned differently for the left or right extremities). In the case of compressive band and hemostatic dressing system with balloon component, when the device is used on the right side, the airline will be pointed distally. When used on the left side, the airline will be pointed proximally.
6. The sheath may be removed at this stage.
7. When applying additional compression in the case of balloon use, inflate the balloon component by attaching the inflation syringe to the balloon airline valve. Syringe volume delivery is preset by position of the plunger within the syringe body prior to airline attachment. Normal range of inflation volume is 6 to 8 ml of air with maximum inflation 15 ml.
8. Alternatively to step 6, the sheath may be removed at this stage.
9. If bleeding is observed from the edge of the hemostatic dressing, add additional compression until bleeding stops. It is recommended, in the case of balloon compression, that additional air be added in 2 ml volume increments.
10. Evaluate arterial patency by using a recommended evaluation procedure (use the reverse Barbeau's test in the transradial procedure: place the plethysmographic sensor on the index finger of the involved upper extremity with the observation of pulsatile waveforms; compress the Ulnar artery at the level of the wrist, and observe the behavior of the waveform. Absence of plethysmographic waveform is indicative of interruption of radial artery flow. If this occurs, the hemostatic compression pressure should be lowered to the point where plethysmographic waveform returns. This is evidence of antegrade radial artery flow).
11. At the recommended band assembly removal time, remove the band compression pressure. In the case of balloon use, remove 2 mls of air every 10 minutes. If bleeding is observed during this time, add sufficient incremental 2 ml compression to restore hemostasis. After 10 minutes, continue incremental 2 ml air volume reduction until all air is removed.
12. Once the compression is removed, and it is confirmed that there is no bleeding, unfasten the compressive band and hemostatic dressing system and press down with moderate to firm digital pressure on the release tab against the patient's extremity. While holding pressure against the release tab, slowly release the hemostatic dressing assembly from the band assembly without disturbing the dressing attachment to the extremity.
13. Remove the band assembly from the extremity leaving hemostatic dressing assembly attached in place on the extremity covering the injury.
14. Dispose of the used band assembly and inflation syringe.
15. Apply a securement dressing to secure the hemostatic dressing assembly in place.
16. Instruct the patient to remove the securement dressing and hemostatic dressing assembly within 24 hours by irrigating with saline or water while gently pulling up on a corner of the hemostatic dressing assembly.

The present invention may or may not include markings to facilitate proper positioning of the hemostatic patch on a puncture injury.

In a preferred embodiment, the compressive band and hemostatic dressing device comprises a positioner assembly, a strap comprising a flexible hinge assembly, a compression plate, and a releasably bound hemostatic dressing. The strap comprising a flexible hinge assembly provides independent movement of the positioner assembly and the releasably bound hemostatic dressing. In this embodiment, the device may further comprise a compressive cushion component on one of the compression plate or strap, and the hemostatic dressing is provided on the compressive cushion component. The hemostatic dressing may comprise chitosan. The compression plate may be substantially flat and located in a pocket provided within the strap. The device may be non-occlusive, such that it does not occlude arteries or veins and helps to prevent complications and injuries related to occlusion.

In another preferred embodiment, the compressive band and hemostatic dressing device of the present invention comprises a strap comprising a flexible hinge assembly, a compression plate, a compressive cushion component, and a hemostatic dressing assembly, a positioner assembly movably connected to a strap by the flexible hinge assembly, and a hemostatic dressing releasably bound, attached, or connected to the compressive cushion component. In this embodiment, the compression plate may be rigid and substantially flat and the compressive cushion component may be located entirely under the compression plate and strap with a flexible hinge assembly. In this embodiment, the compression plate may be located in a pocket provided within the strap or located on a surface of the strap. In this embodiment, the positioner assembly may be flexible or semi-rigid with a fixed curve profile. In this embodiment, the hemostatic dressing may comprise chitosan, such as a phase-separated, freeze-dried chitosan acid dressing, including an acid salt comprising one of acetic, lactic, glycolic or citric acid. In this embodiment, the compressive cushion component may be attached to one of the compression plate and the strap. In this embodiment, the compressive cushion component may be attached along only one edge to a surface that is off-center relative to the compression plate. This embodiment may provide a device that is non-occlusive and a hemostatic dressing assembly that is attached to one of the compression plate, the strap, and the compressive cushion component depending on its configuration.

Benefits of using the present invention may be realizing using the various device embodiments and methods described herein. One preferred method involves using a compressive band and hemostatic dressing device, such as those described above, and includes the step of leaving the hemostatic dressing in place after hemostasis is achieved and the positioner assembly, strap with a flexible hinge assembly, compression plate, and compressive cushion component are removed from a puncture site. This method may further including the steps of placing the device with the aid of the strap flexible hinge assembly to independently move the positioner assembly and the hemostatic dressing, and applying compression, preventing artery and vein occlusion. This method may further comprise the step of locating the compressive cushion component entirely under the compression plate and strap so that it is not subject to direct compression by the positioner assembly. Limiting the location of the compressive cushion component so that it is not directly compressed by the positioner assembly facilitates proper and safe functioning of the compressive component by avoiding interaction between the positioner assembly and the compressive cushion component that might otherwise impinge or obstruct its function.

### BRIEF DESCRIPTION OF THE DRAWINGS

The invention may be more clearly understood from the following description of an embodiment thereof, given by way of example only, with reference to the accompanying drawings, in which:

FIG. 1 provides a drawing of a preferred embodiment of the compressive band assembly and hemostatic dressing system of the invention (with balloon component) in plan view. The position of the airline connection to the balloon bladder and the protruding release tab are shown as example positions in the compressive band assembly and hemostatic dressing system of the invention. In FIG. 1, the position of the airline connection to the balloon bladder may be opposite to the protruding release tab. Not shown in FIG. 1 is an alternative embodiment where the airline connection to the balloon bladder is located on the same side as the protruding release tab, in which case the airline connection may be offset relative to the center of the balloon to avoid interference with ease of access to the release tab or the introducer sheath. The hemostatic dressing and tab release as shown in the drawings are with rounded corners, the corners may be square or a combination of square and rounded.

FIG. 2 provides a drawing of the preferred embodiment presented in FIG. 1 in side view wherein the protruding release tab is not shown and without the airline connection tubing extending from the compressive band assembly.

FIG. 3 provides a drawing of the preferred embodiment presented in FIG. 1 in a perspective view of finally assembled band and hemostatic dressing.

FIG. 4 provides drawings of the preferred embodiment presented in FIG. 1 in an exploded perspective view of individual band components.

The balloon component assembly as provided in FIGS. 5a, 5b, 5c and 5d wherein the open balloon bladder body is formed of a single sheet of material that is folded over a central fold line to provide for a final closed balloon component body by joining, welding or gluing together of opposing edges. This method of join, weld or glue closure in the preferred embodiment drawings of FIGS. 5a, 5b, 5c and 5d, in no way limits other closed balloon component embodiments, including those which may be formed of joining, welding or gluing together of two separate sheets of balloon body material or the joining closed of the two ends of an extruded tube of the material of the balloon bladder body. An integral closed balloon bladder comprising a plastic material may be formed with no join, weld or glue connection, other than the connection to the airline. Such a balloon bladder, although not described in full here, could also be part of the balloon component of the compressive band and hemostatic dressing system of the invention.

FIG. 5a provides a preferred embodiment of the assembled balloon component.

FIG. 5b provides a preferred embodiment of the balloon component of FIG. 5a in individual, non-assembled components.

FIG. 5c provides a preferred embodiment of the assembled balloon component with airline connector sleeve containing a wing edge profile.

FIG. 5d provides a preferred embodiment of the balloon component of FIG. 5c in individual, non-assembled components.

FIG. 5e provides a drawing of a preferred “syringe pump” embodiment for adding to or removing air from the balloon assembly.

FIG. 5f provides a drawing of a preferred pressure gauge test system.

FIG. 6a presents a photographic image (viewed from above) of the top side of the packaging support insert (with syringe recess) that is used to support and protect the compressive band and hemostatic dressing system of the invention within its packaging system during distribution and storage.

FIG. 6b presents a photographic image (viewed from above) of the under-side of the packaging support insert (with syringe recess) of FIG. 6a.

FIG. 6c presents a photographic image (viewed from above) of the packaging support insert of FIG. 6a with compressive band and hemostatic dressing system with balloon component.

FIG. 7 provides a drawing of a preferred embodiment of the compressive band and hemostatic dressing system of the invention (without compressive cushion component) in plan view.

FIG. 8 provides a drawing of the preferred embodiment of FIG. 7 in side view.

FIG. 9 provides drawings of the preferred embodiment of FIG. 8 showing the flexible joint between the positioner assembly and the compression plate at 180°, 210°, 150° and 135°.

FIG. 10 provides a drawing of a preferred embodiment of the compressive band and hemostatic dressing system of the invention with a preformed compressive cushion component in side view.

FIGS. 11a, 11b, and 11c provides black and white images of thumb and forefinger pressure over the positioner assembly and the compression plate joined by the flexible hinge element providing for desirable light to moderate pressure application over the hemostatic dressing.

FIG. 12 details the results of preclinical sheep model testing of chitosan HemCon Bandage dressing.

FIG. 13 presents a graphical plot of results of average load in grams and consistency (error bars) in loading against a rigid, 2 inch diameter, pipe fixture following discrete volume balloon addition and volume removal (milliliters of air).

FIG. 14 provides a Table of outcome of pressure (psi) testing after initial charging of 7 psi of air into balloon component for 5, 10, 20, 30, 60, 90 and 120 minutes in nine (9) randomly selected successful (passed initial 10 minute test) development lot balloon components.

FIG. 15 provides a Table of results demonstrating comfort, and load change of the compressive band system around non-injured, volunteer subject (N=5) wrists for a period of 120 minutes after initial loading to a target load of 700 g.

FIG. 16 shows load change plotted against time of the compressive band system around non-injured, volunteer subject (N=5) wrists for a period of 120 minutes after initial loading to a target load of 700 g.

FIGS. 17a, 17b, 17c, 17d, and 17e depict various embodiments of the compressive band and hemostatic dressing system of the present invention. FIG. 17a provides a top plan view and a side exploded view of an embodiment comprising an asymmetric balloon assembly and a strap with a flexible hinge assembly comprising a pocket holding the compressive plate, and including a hemostatic dressing assembly. FIG. 17b provides the side view of an embodiment comprising an asymmetric balloon assembly and a strap with a flexible hinge assembly comprising a pocket holding the compressive plate, and including a hemostatic dressing assembly, as configured for application. FIG. 17c provides a top plan view and a side view of an embodiment comprising a symmetric balloon assembly and a strap with a flexible hinge assembly comprising a pocket holding the compressive plate, and including a hemostatic dressing assembly. FIG. 17d provides a top plan view and a side view of an embodiment comprising a symmetric centrally attached pillow balloon assembly and a strap with a flexible hinge assembly comprising a pocket holding the compressive plate, and including a hemostatic dressing assembly. FIG. 17e provides a top plan view and a side view of an embodiment comprising a laterally attached balloon assembly, which attaches to the strap and is located under the compression plate, and a strap with a flexible hinge assembly comprising a pocket holding the compressive plate, and including a hemostatic dressing assembly.

FIGS. 18a and 18b provide photographic images of a prototype embodiment of the present invention similar in construction to the compressive band and hemostatic dressing system depicted in FIG. 17e. FIG. 18a depicts this embodiment without the hemostatic dressing assembly; meanwhile, FIG. 18b includes this embodiment with a hemostatic dressing assembly.

### DETAILED DESCRIPTION

The present invention provides compressive band and hemostatic dressing systems and methods for providing simple to apply, fast, non-occlusive bleeding control, vascular injury treatment, and injury protection comfortably and reliably after a percutaneous vascular access procedure.

Throughout this description it is to be understood that reference identifiers applied in relation to a specific figure also relate to like or similar features depicted in other figures regardless of whether such figure features are specifically called out in connection with each figure below.

The compressive band and hemostatic dressing systems 200 of the invention may comprise all, or a selection of the following elements: a positioner assembly 10, a compressive cushion component 30, a compression plate 50, a strap 70 with a flexible hinge assembly 72, and a hemostatic dressing 90. Preferred embodiments described herein include the compressive band and hemostatic dressing systems of the invention with and without the compressive cushion component 30.

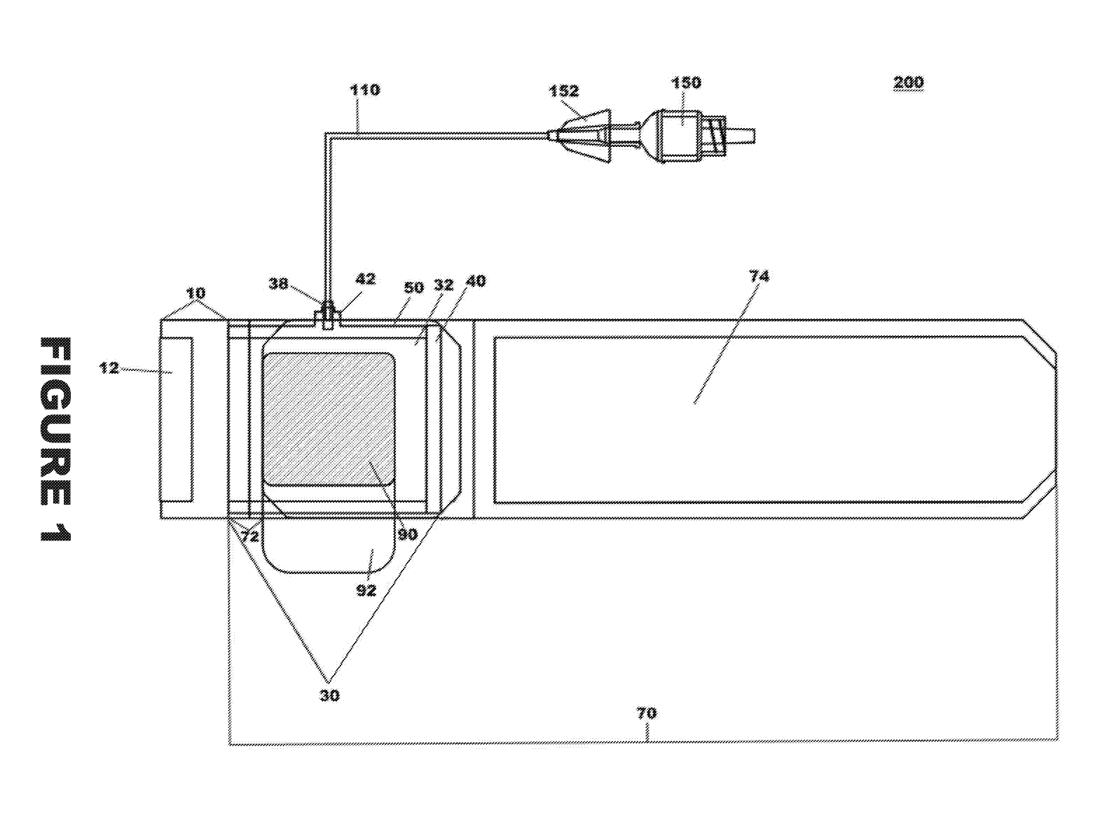

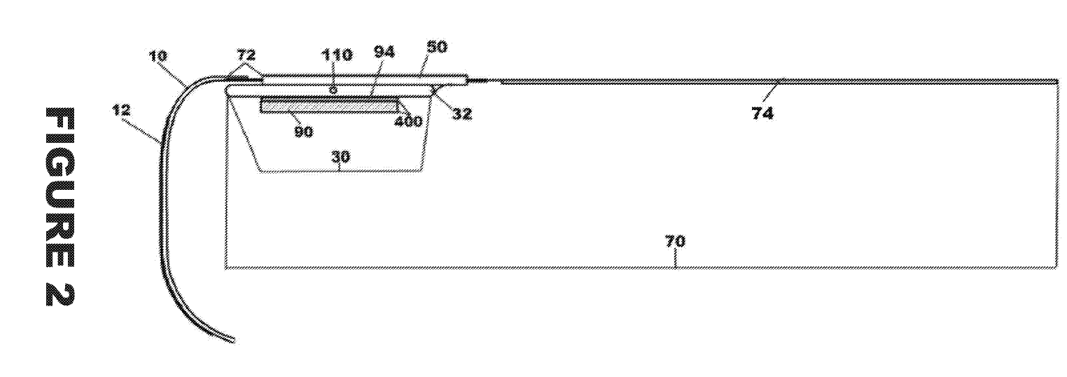

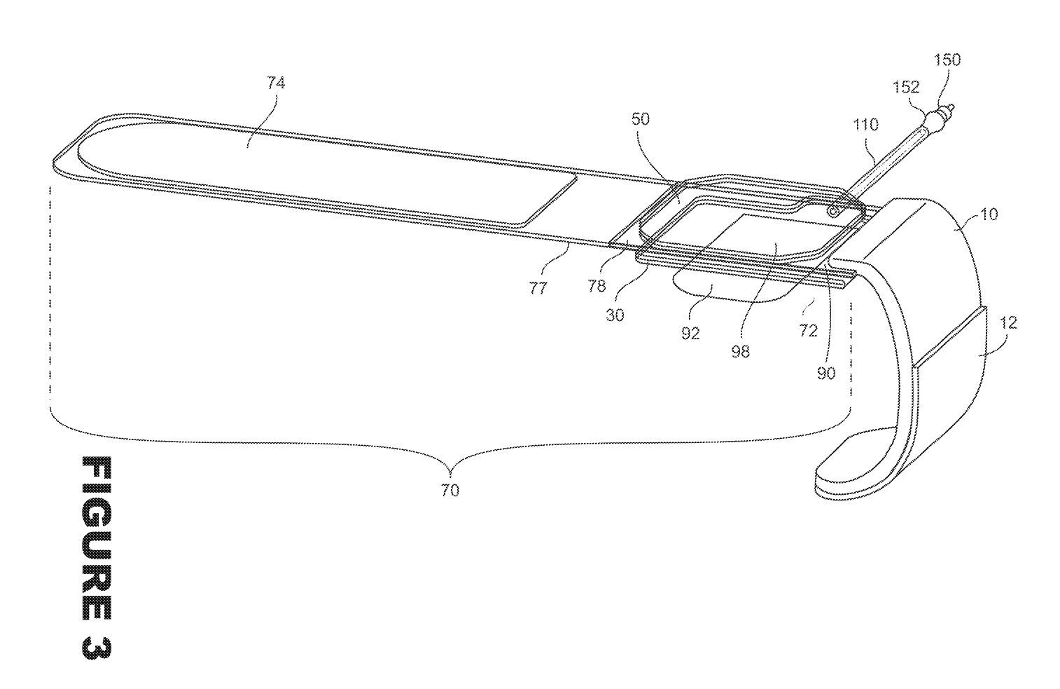

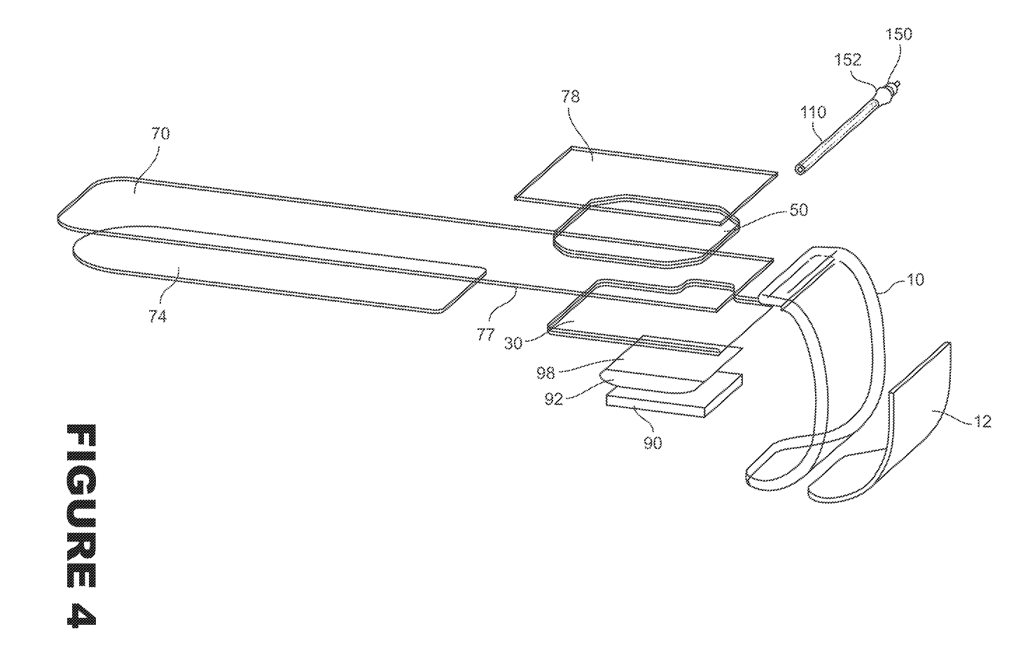

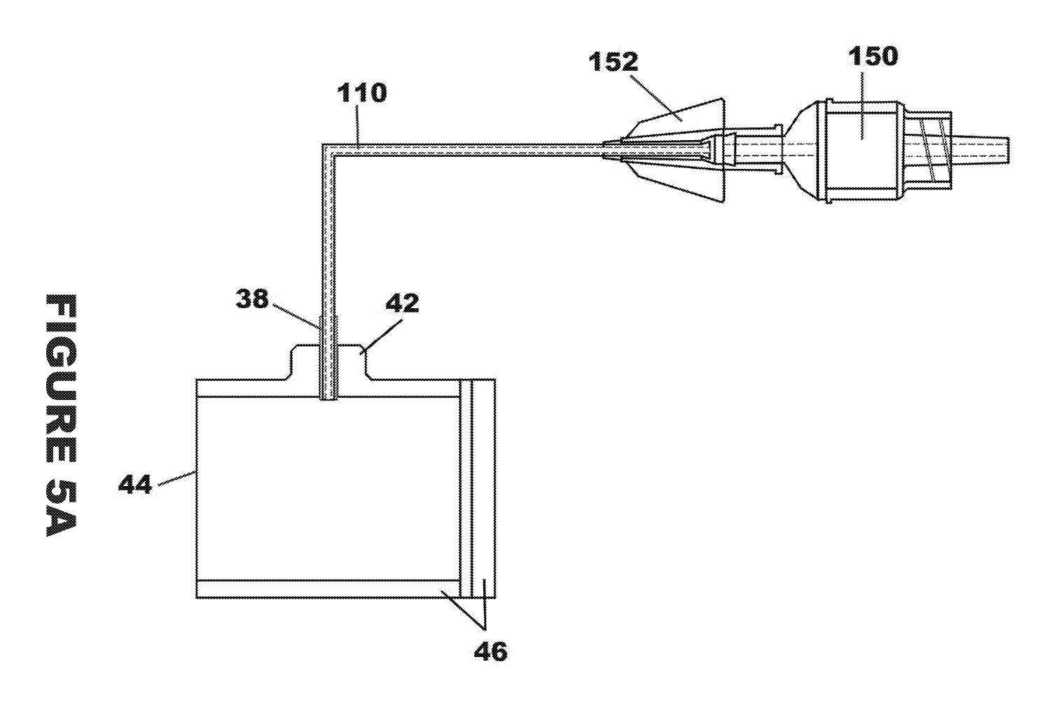

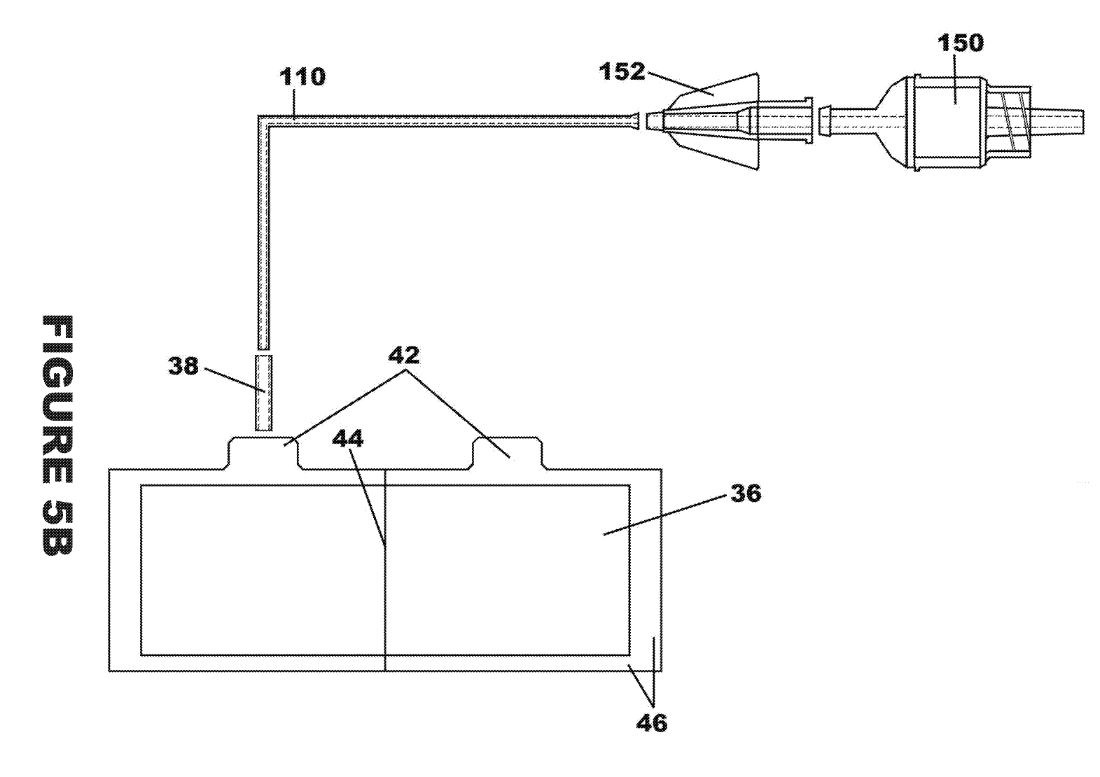

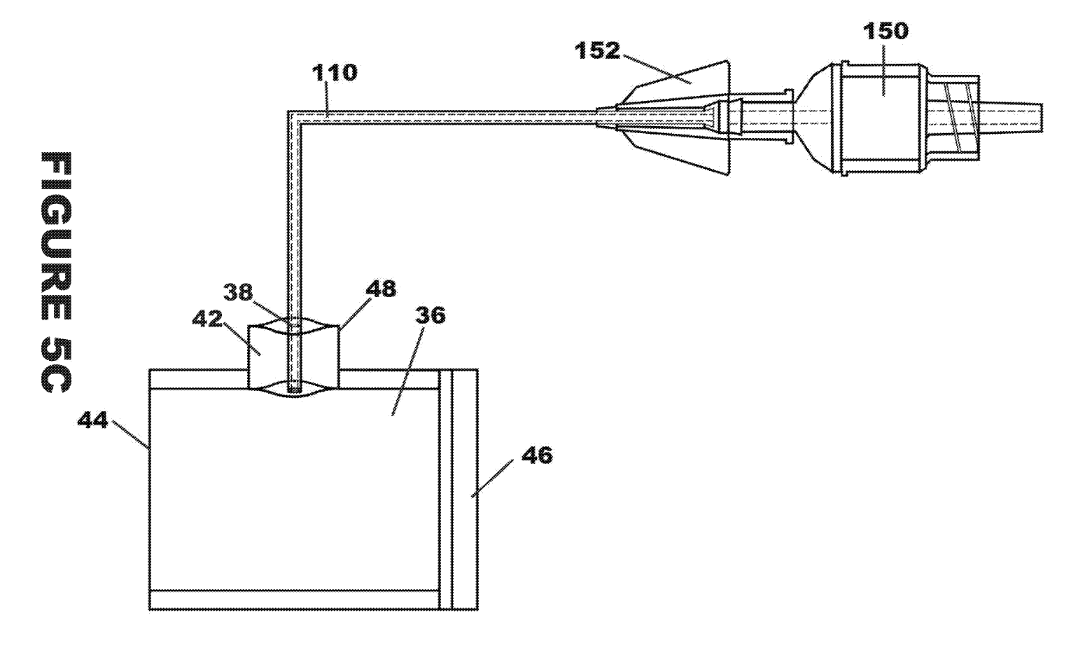

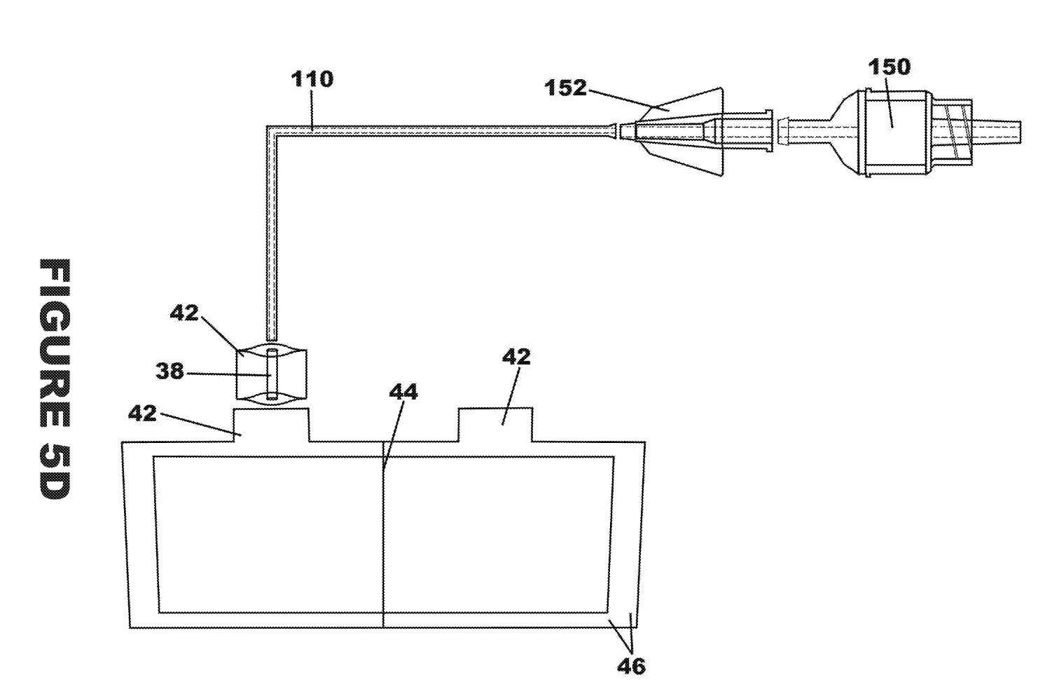

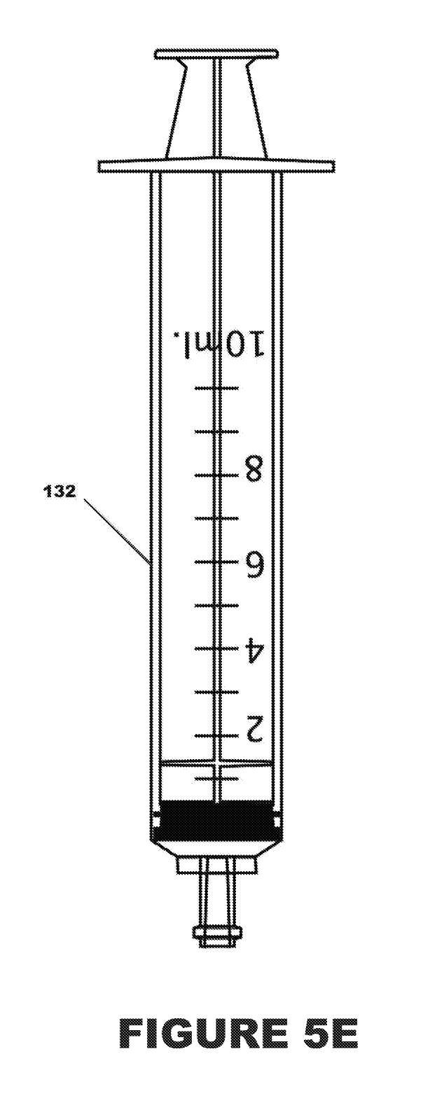

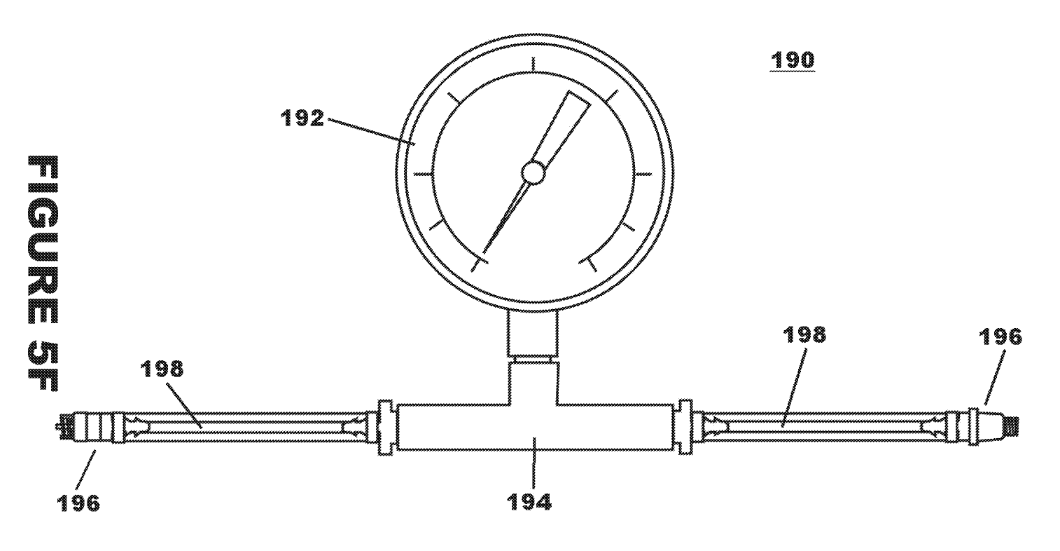

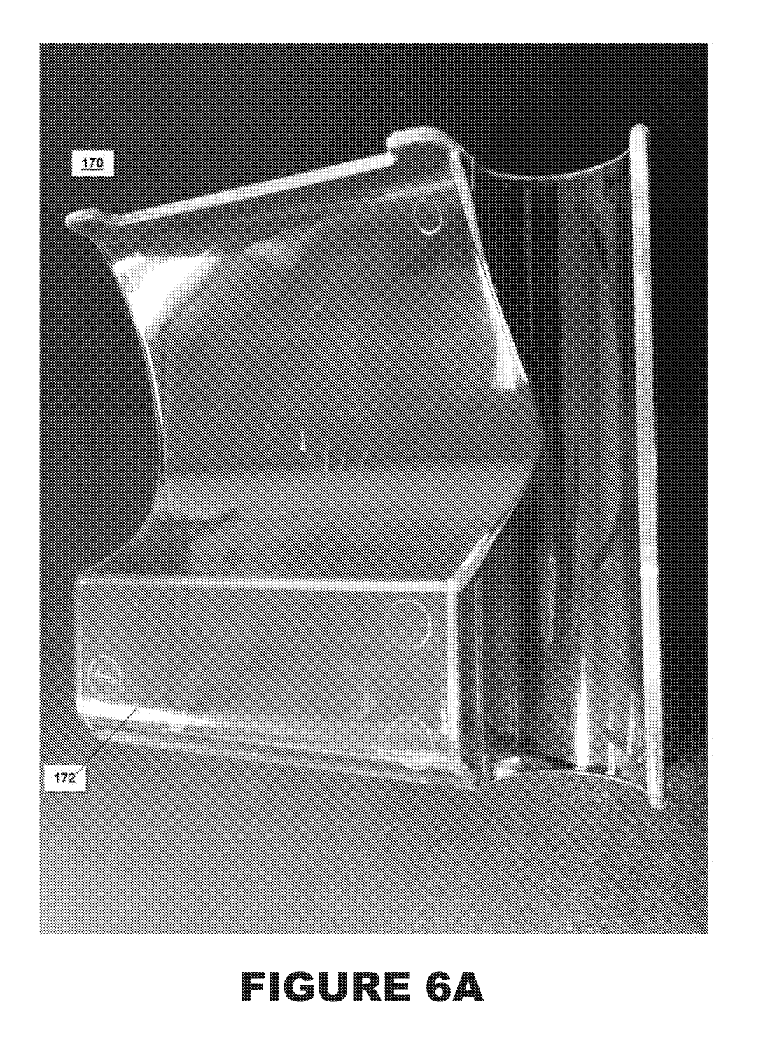

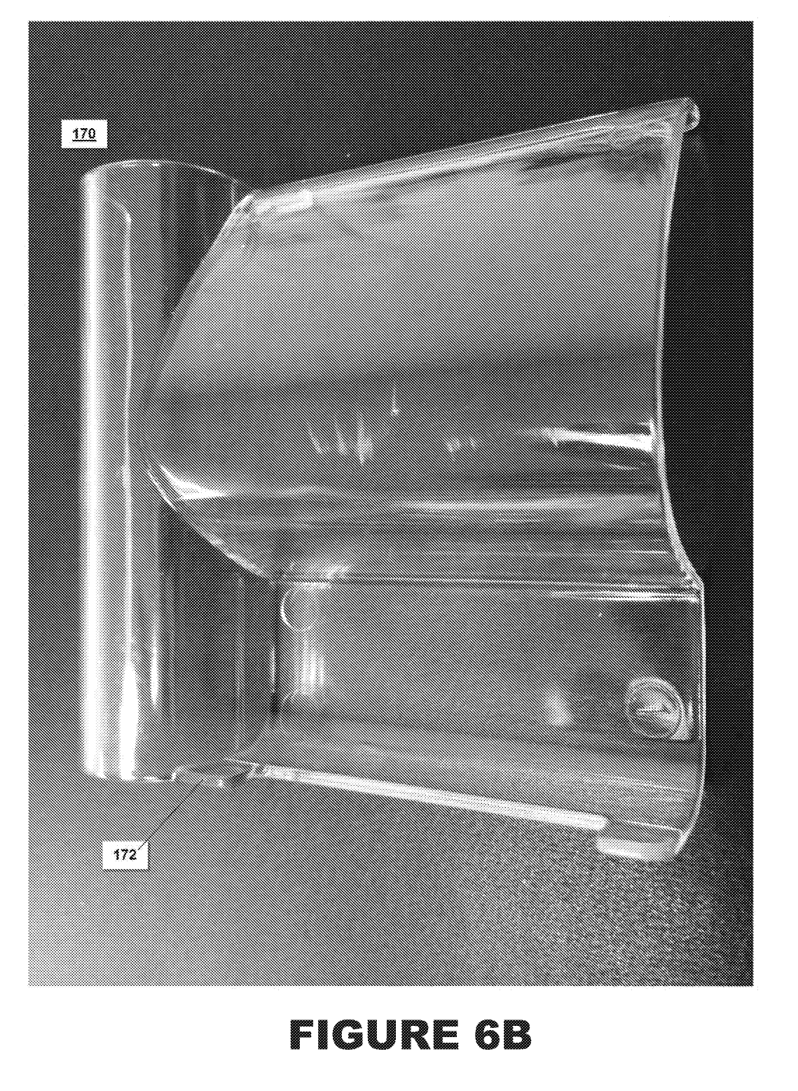

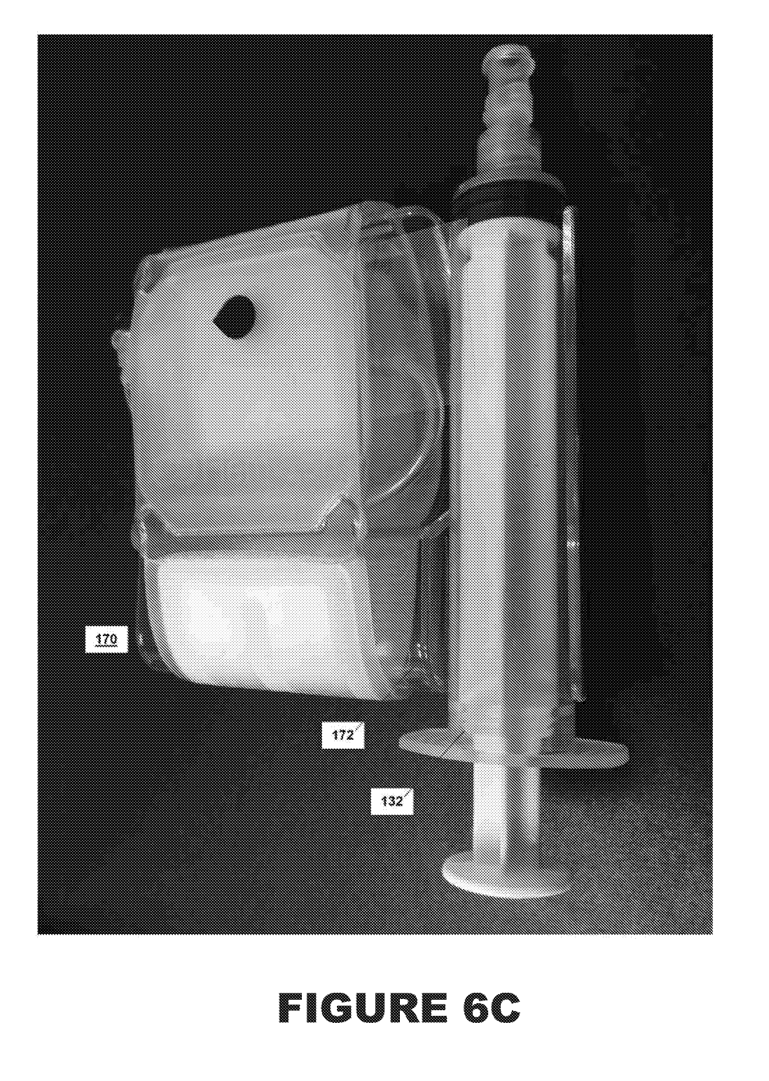

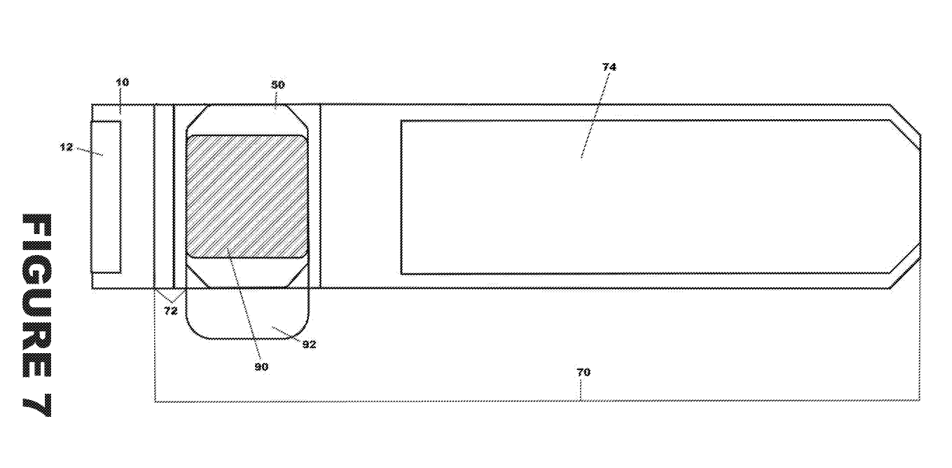

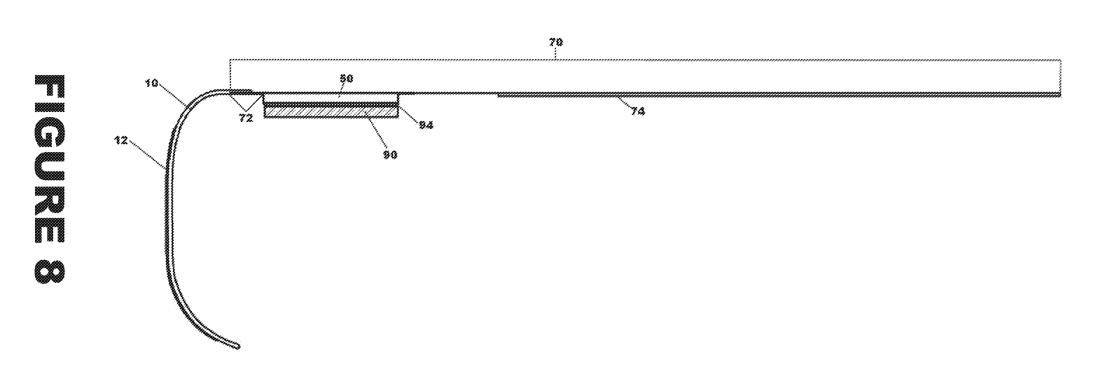

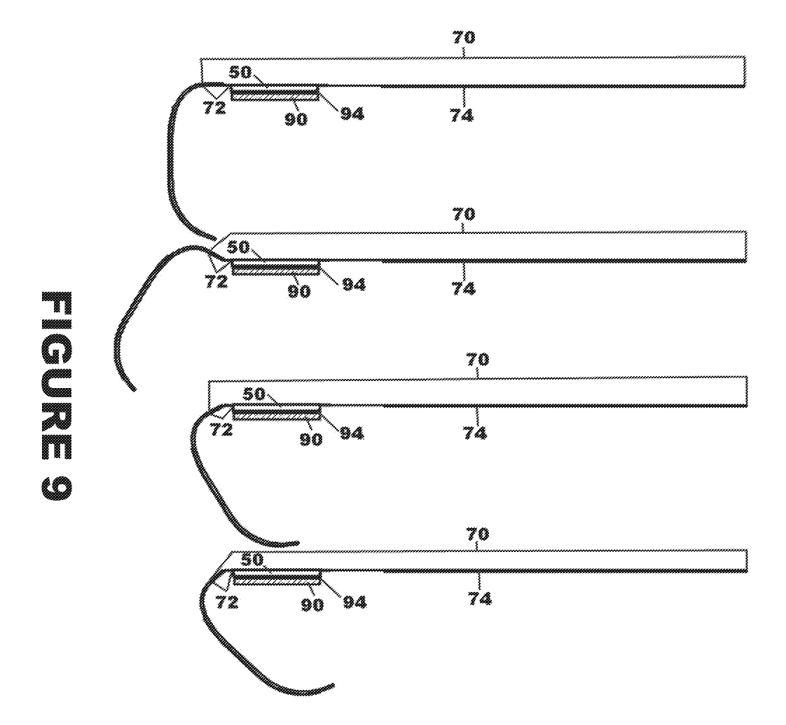

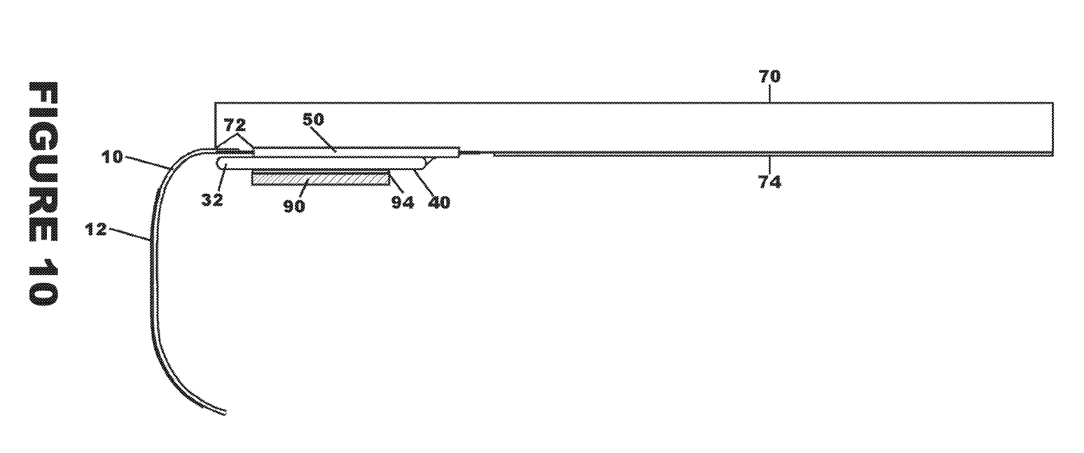

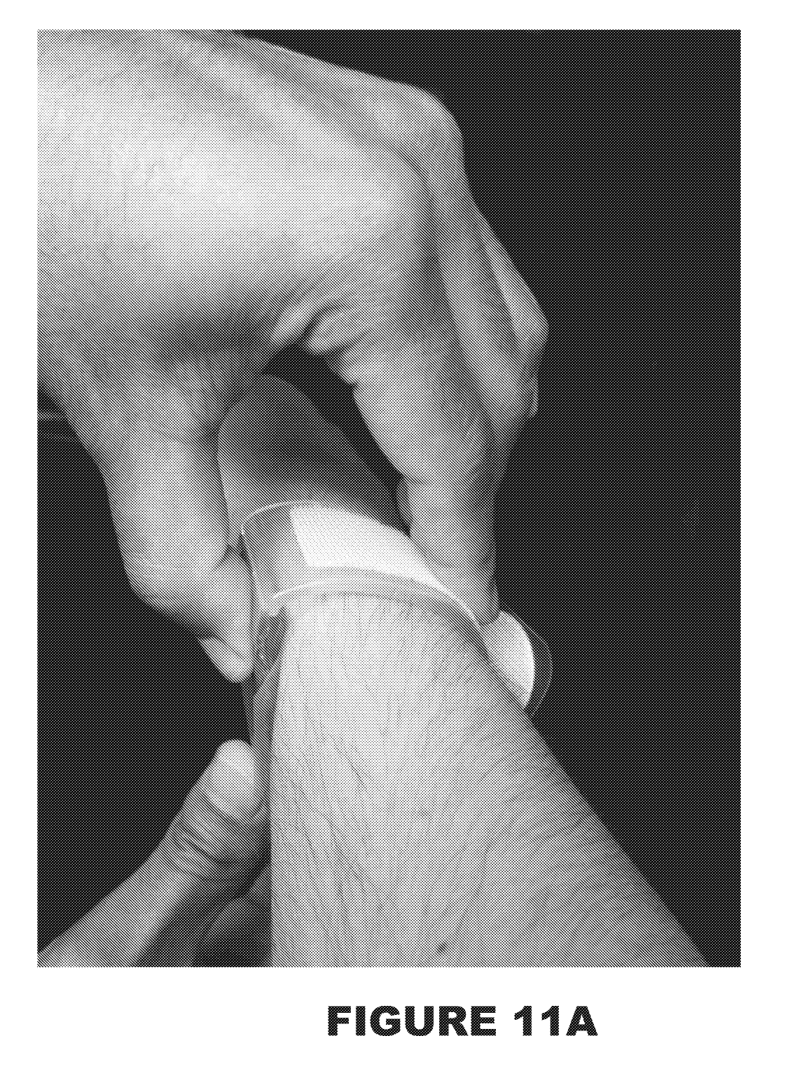

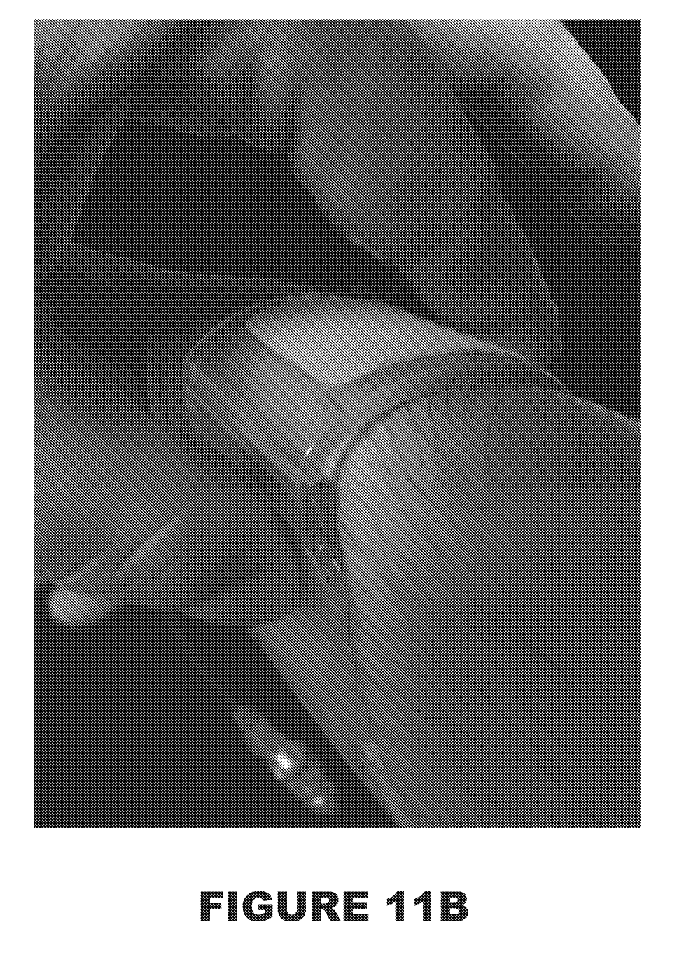

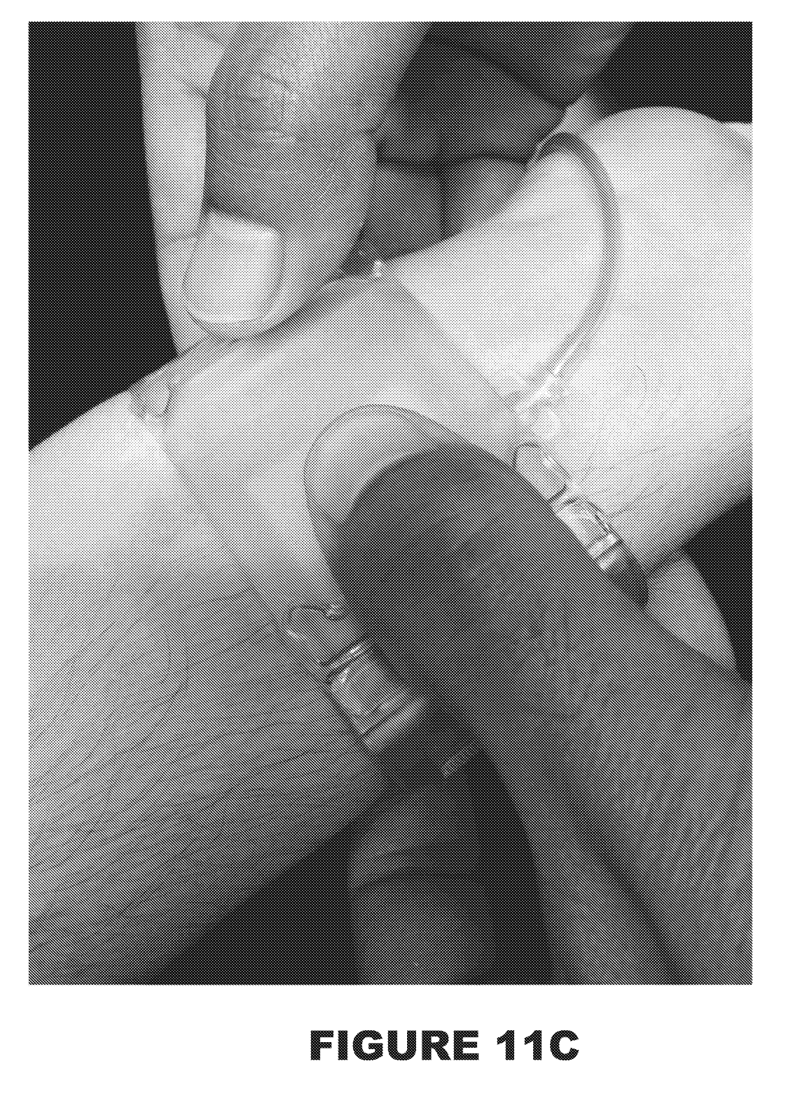

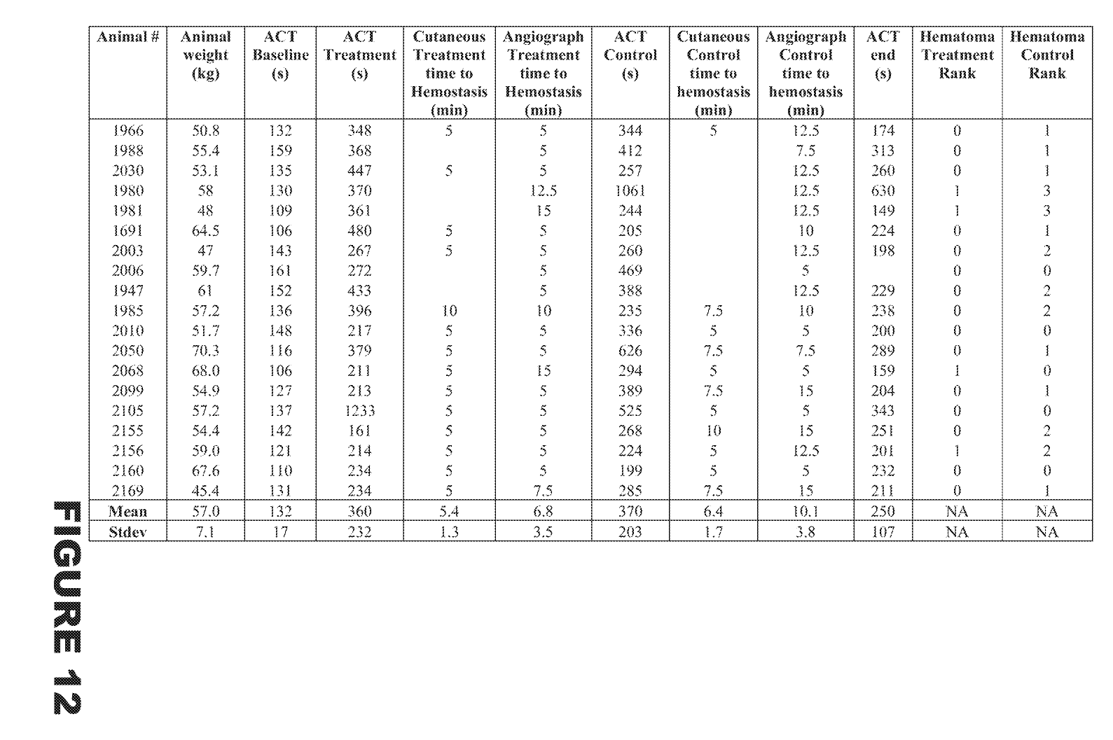

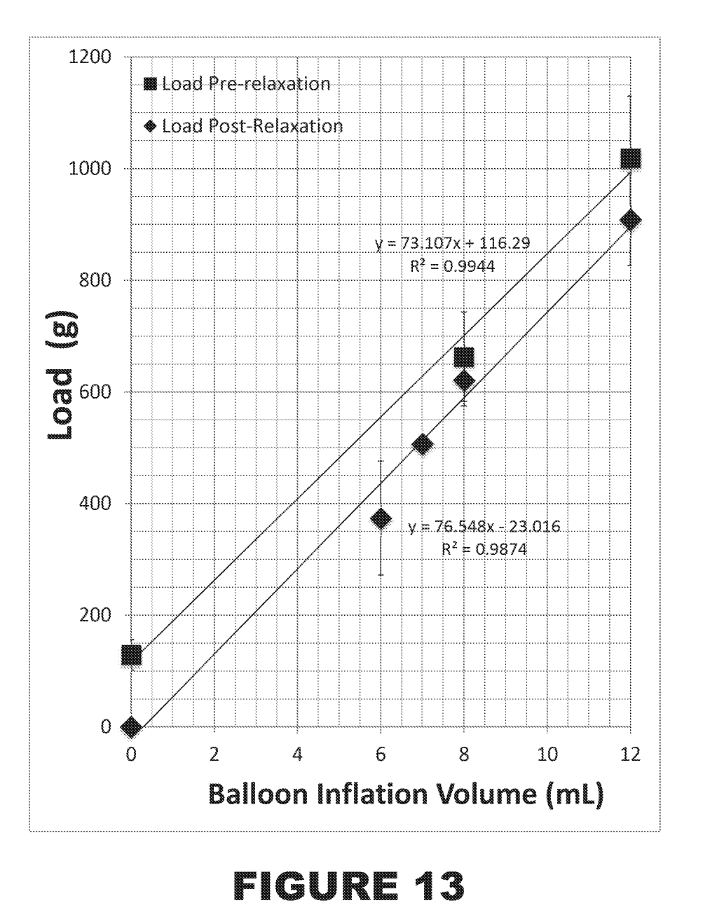

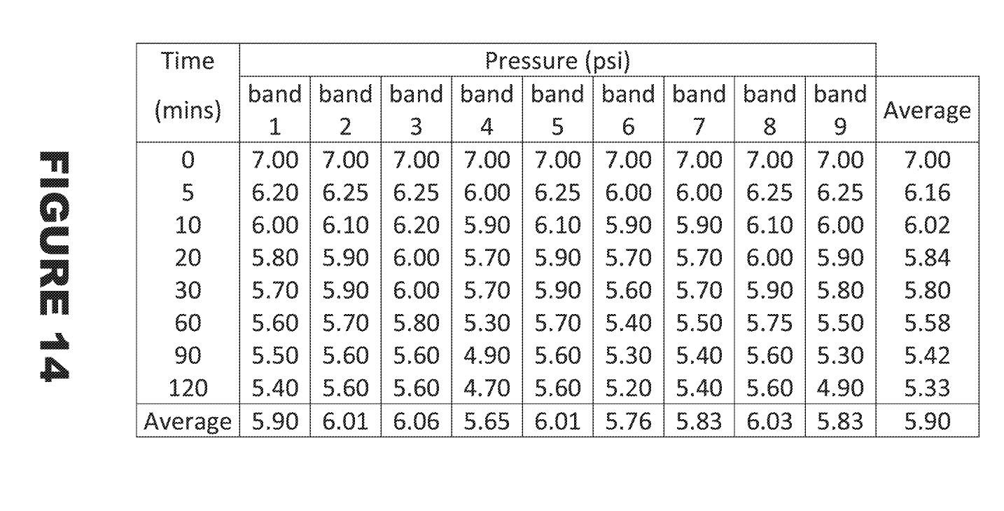

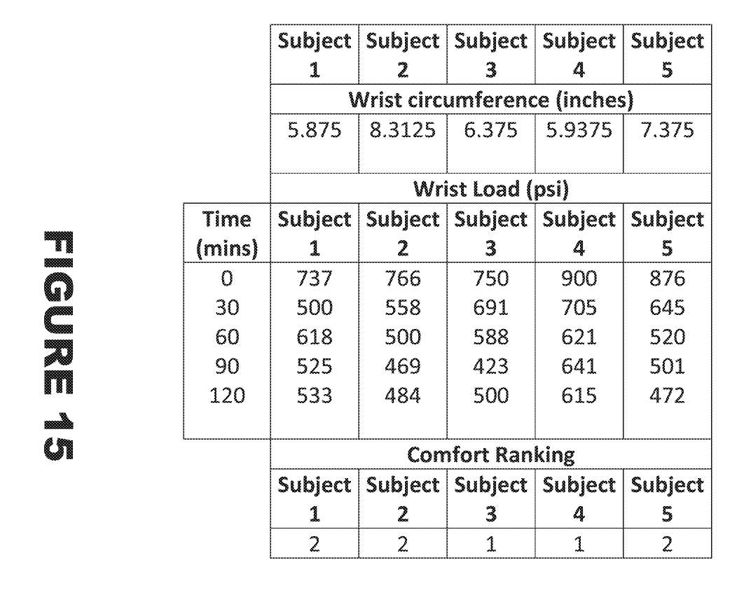

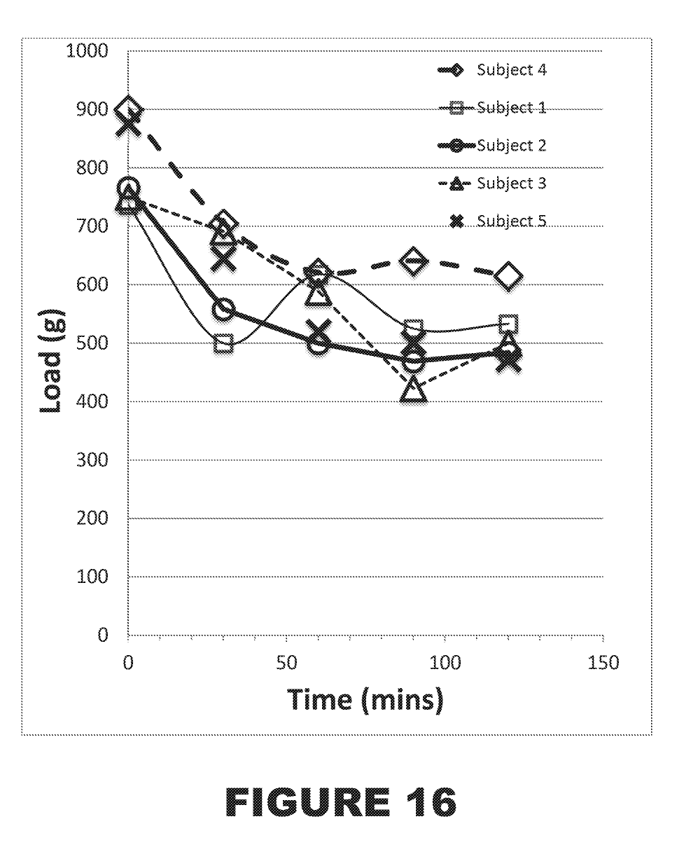

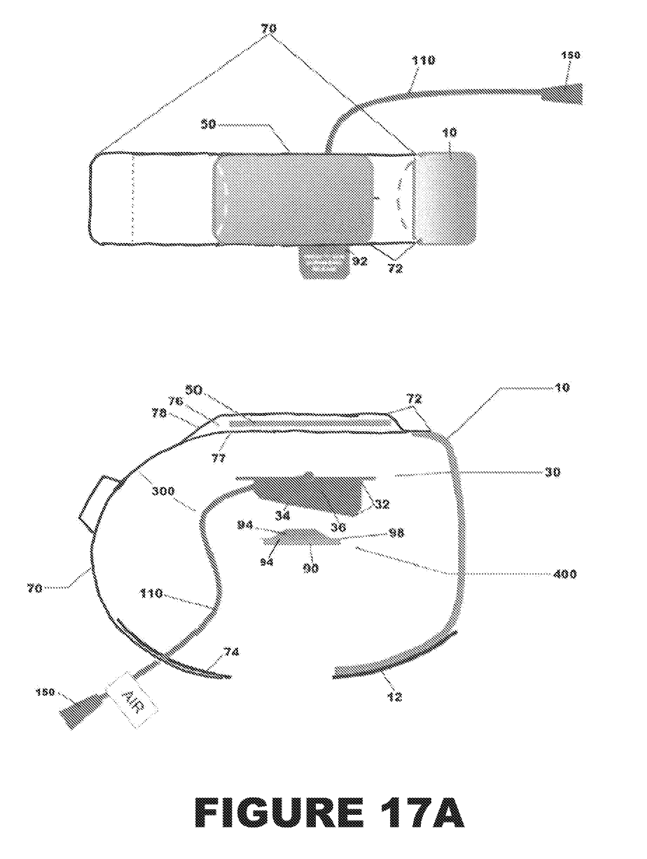

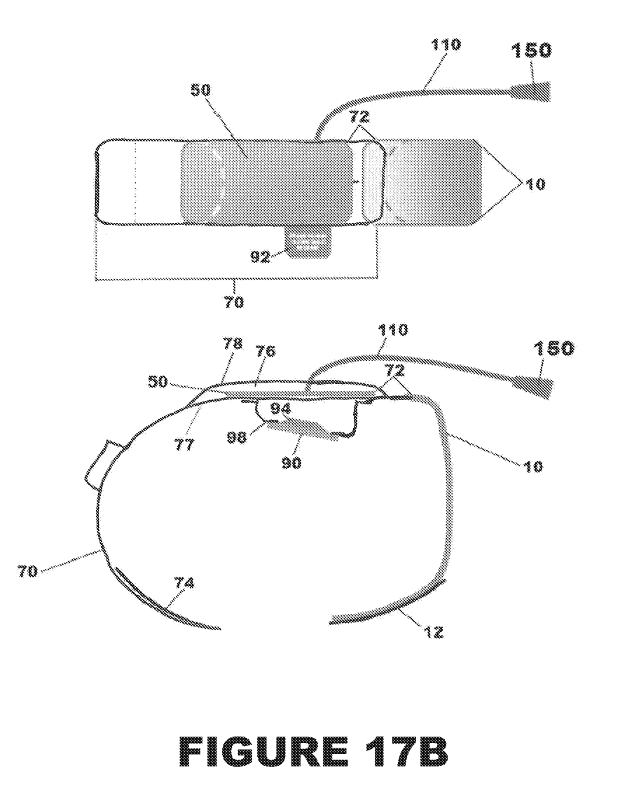

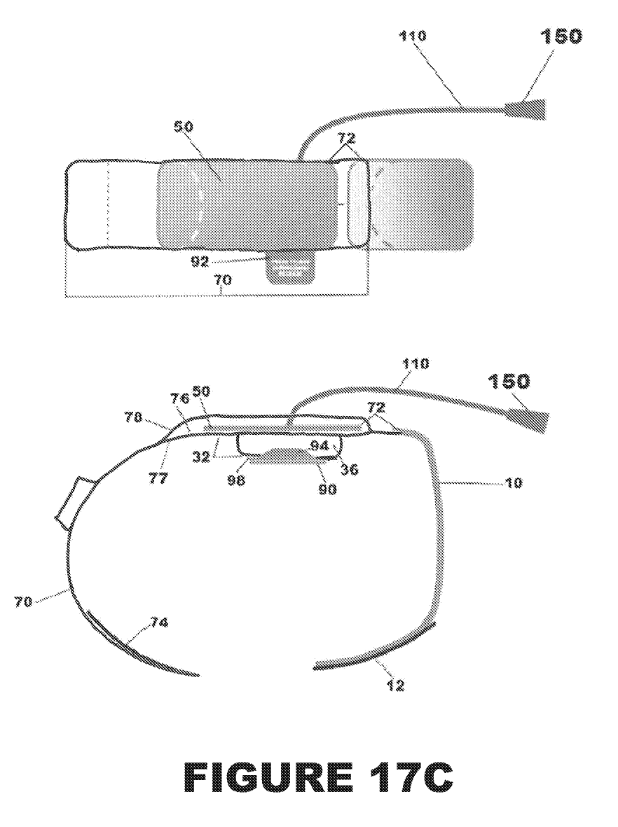

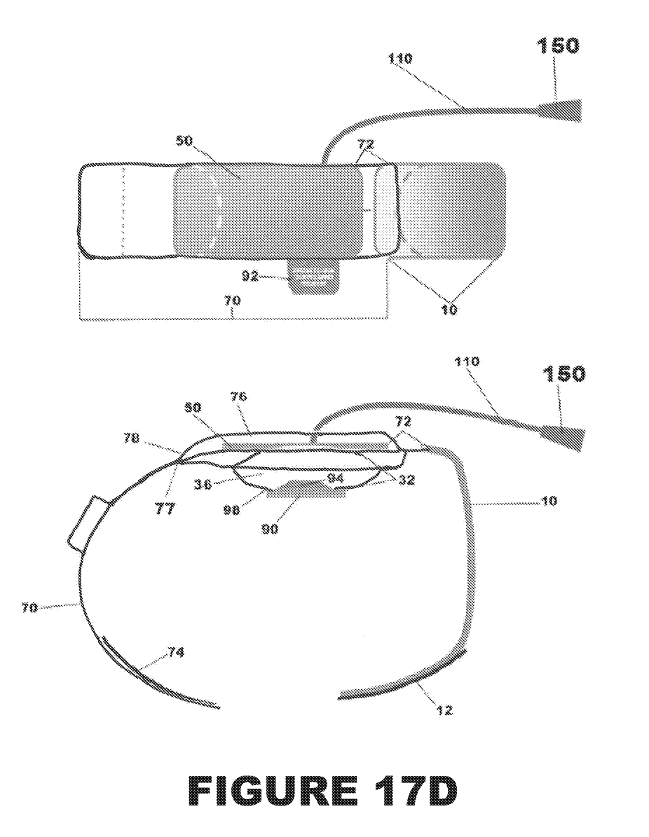

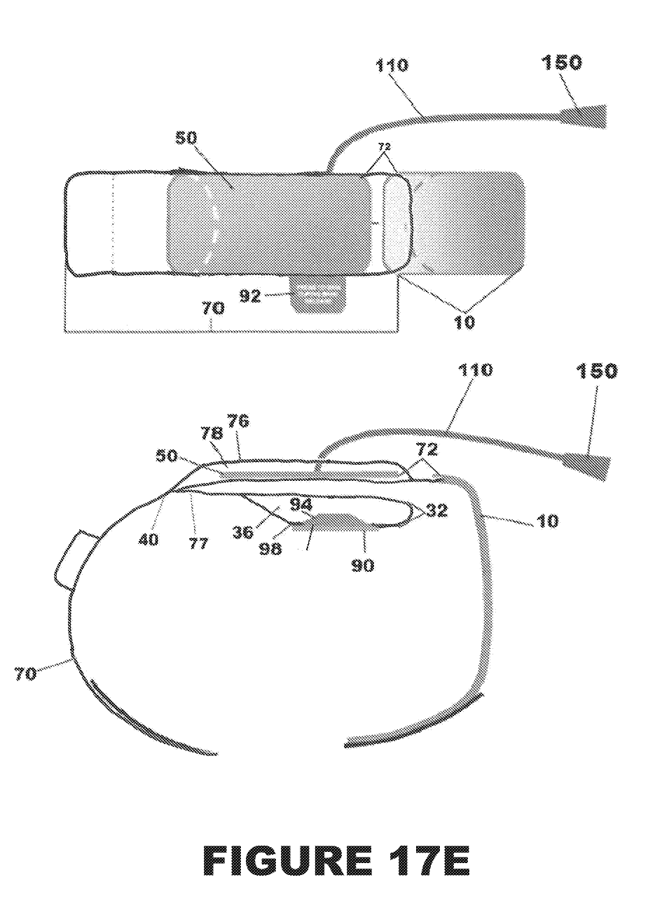

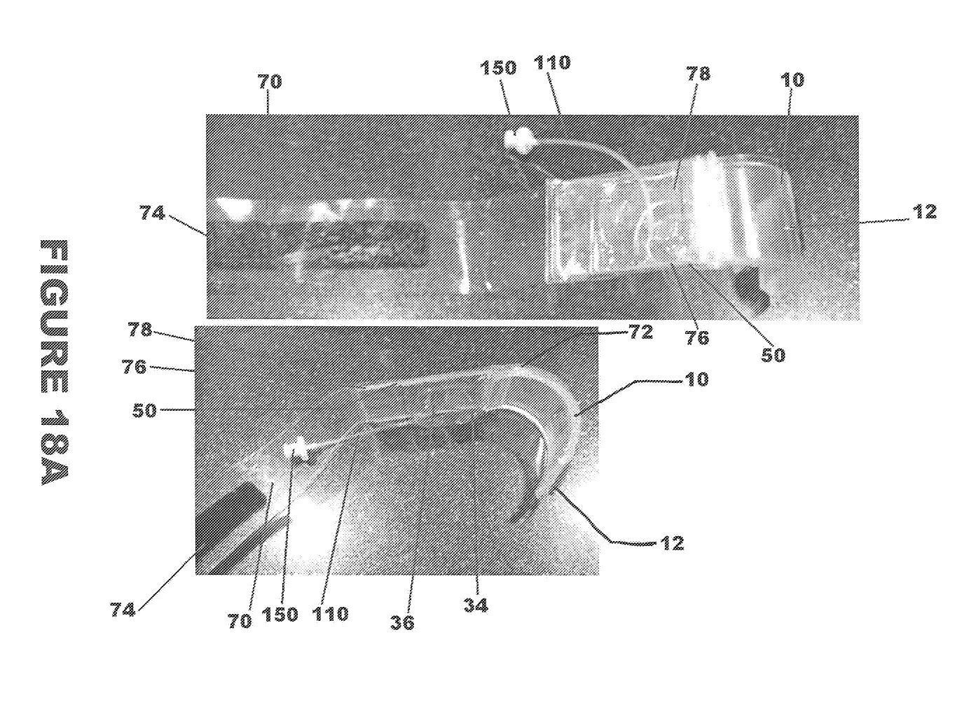

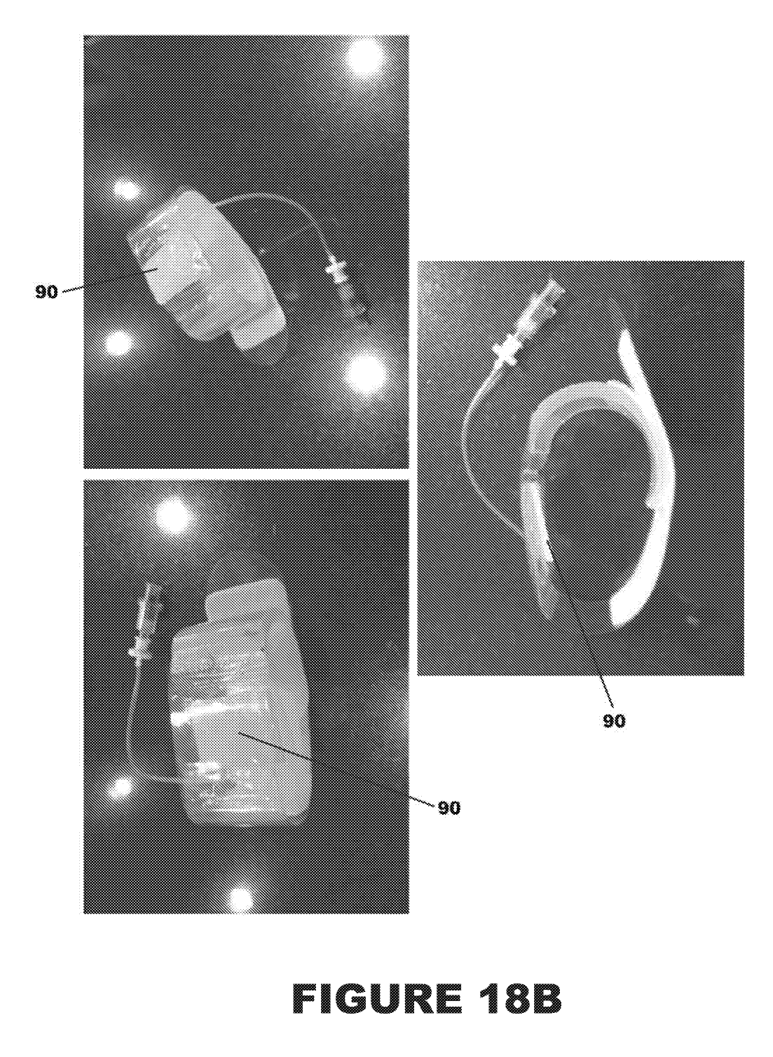

FIGS. 1, 2, 3, and 4 depict a preferred embodiment of the compressive band and hemostatic dressing systems 200 that include a compressive cushion component 30 as a balloon component 32. Here the positioner assembly 10 is flexible or semi-rigid with fixed curve profile, the compression plate 50 is rigid with fixed flat or planar profile configured for placement immediately over the injury and hemostatic dressing. Compression plate 50 is separate from, but flexibly connected by way of flexible hinge connector 72 to the positioner assembly 10. Strap 70 comprises the flexible hinge connector 72, which is located as part of and between the strap 70 pocket 76 (which holds the compression plate 50) and the positioner assembly 10.

The compressive cushion component 30 is provided below the compression plate 50 held in strap 70 pocket 76. The compressive cushion component 30 is a singular compressive cushion without a supportive or protective frame, tray, case, etc., and is symmetric in shape. The compressive cushion component 30 comprises a balloon component 32 that can be conveniently inflated or deflated with a fluid as needed to achieve a desired compression over the hemostatic dressing 90. Balloon component 32 is attached to the injury-facing surface of strap 70 below the compression plate 50 via weld seam 40. The balloon component 32 includes a feed tube 110 that connects the interior of the balloon bladder 36 with a pump (not shown here, see, e.g., the syringe 132 shown in FIG. 5e) to regulate pressure (at or near ambient temperature) within the balloon component 32. The feed tube 110 may connect to the pump through a valve 150. The balloon component 32 includes a dressing support surface 34, and airline connector sleeve 38, and a neck 42.

The pressure within the balloon component 32 is maintained by addition or removal of a controlled amount of fluid from the pump, such as a syringe 132 (see e.g., FIG. 5e) that is able to connect with the valve 150 to provide an airtight connection, and that is able to deliver or remove consistent amounts of fluid over a pressure range of 0 to 14 pounds per square inch. Here, valve 150, when it is connected to the pump, enables the pump to add or remove discrete and consistent amounts of fluid through the valve 150 to and from the balloon component 32; however, when the pump is disconnected, the valve 150 provides for maintenance of the amount of fluid within the sealed balloon component by not allowing transfer of fluid through the valve 150. The balloon component 32 is able to maintain consistent and reliable internal pressure with addition of a fixed amount of air into the empty balloon bladder to within ±35% over 120 minutes under ambient temperature and pressure conditions and under initial internal pressure of 7±1 pounds per square inch, or more preferably to within ±25% over 120 minutes under ambient temperature and pressure conditions and under initial internal pressure of 7±1 pounds per square inch, and most preferably to within ±20% over 120 minutes under ambient temperature and pressure conditions and under initial internal pressure of 7±1 pounds per square inch.

Each balloon component 32 may be individually tested for internal pressure consistency and reliability prior to acceptance for use in the compressive band system 300 and hemostatic dressing system 400. Inclusion of balloon component 32 as part of the compressive band system 300 and hemostatic dressing system 400 as packaged and stored should not compromise balloon component 32 reliability and consistency (see, for example, FIGS. 6a, 6b, and 6c which depict the compressive band and hemostatic dressing system (with balloon component) including the packaging support insert 172).

The compression plate 50 is rigid and flexibly held within a pocket 76 of the strap 70, which includes the flexible strap hinge mechanism 72, such that the compression plate 50 is movable relative to the positioner assembly 10. Pocket 76 is formed as part of the strap 70 by the pocket and hinge material overlay 78 which is attached to, and forms an integral part of, strap 70 and the flexible strap hinge assembly 72. Accordingly, strap hinge assembly 72 comprises and is reinforced by the material of strap 70 and the pocket and hinge material overlay 78. Strap 70 also includes in a region opposite the flexible strap hinge assembly 72 a strap securement element 74. Here, the securement element 74 is a loop portion of a hook and loop closure. In a preferred embodiment, the positioner assembly securement element 12 on the positioner assembly 10 mates with the securement element 74 on strap 70 to provide for firm and reliable compressive band and hemostatic dressing 90 securement around an extremity such as an arm or a leg. For example, the positioner assembly securement element 12 on the positioner assembly 10 can comprise a hook closure to mate with the strap securement element 74 comprising a loop closure on strap 70.

Here, one or more of the compressive cushion component 30, compression plate 50, and strap 70 may be transparent or semi-transparent; meanwhile, the positioner assembly 10 and hemostatic dressing 90 are non-transparent or opaque. The hemostatic dressing 90 may become partially translucent when wet with blood or may wick a small amount of blood to the caregiver observation side of the hemostatic dressing 90 surface.

Here, the hemostatic dressing 90 is provided immediately under the flat portion of the compression plate 50, with one edge of the hemostatic dressing 90 aligning parallel to the flexible hinge mechanism 72. The hemostatic dressing 90 is supported on the injury-facing surface 34 of the compressive cushion component 30. In alternative embodiments where the compressive band and hemostatic dressing system is provided without a compressive cushion component (depicted, for example, in FIG. 7), the hemostatic dressing 90 is supported on the injury-facing surface 77 of the strap 70 or compression plate 50. The hemostatic dressing 90 is removable or detachable from the compressive band.

The hemostatic dressing 90 may comprise chitosan salts, derivatized chitosan or other mucoadhesive hemostatic agents that, when wet, adhere to, seal, and protect tissue and mucosa injuries. In a preferred embodiment, the hemostatic dressing 90 comprises mucoadhesive chitosan and is unbound, separated, or detached from its compressive band upon removal of the positioner assembly 10, compressive cushion component 30 (when used), compression plate 50, and strap 70 from an injury site. In a preferred embodiment, the hemostatic dressing 90 includes a tab 92 protruding from the edge of the non-injury facing side of the hemostatic dressing 90. The non-injury facing side of the hemostatic dressing 90 attaches to the compressive band assembly by adhesive 94 (not depicted). The tab 92 is permanently and securely attached to the non-injury facing side of the hemostatic dressing 90 surface, and may include but is not limited to polyurethane, polyester, polyethylene, or polypropylene transparent film 98 attached to the non-injury facing side of the hemostatic dressing 90 surface. Tab 92 extends the film 98 beyond one edge of the hemostatic dressing 90. The tab 92 acts as a manually accessible support feature during practice of the invention to allow, by single or double finger pressure on the tab 92 against the patient, leaving the hemostatic dressing system 400 (at least hemostatic dressing 90 and tab 92) in place against the percutaneous access injury, while at the same time allowing removal of the positioner assembly 10, compressive cushion component 30 (when used), compression plate 50, and strap 70 (collectively, the compressive band system 300) from injury site and patient. The hemostatic dressing 90 subsequently is secured in place over the injury by a standard procedure to be removed as desired. In a preferred embodiment, the hemostatic dressing 90 is composed of a protective, antibacterial, wound healing, hemostatic, mucoadhesive, chitosan composition which is left in place for at least 12 hours and then is subsequently removed by the patient by applying running water over the chitosan dressing until the mucoadhesive attachment of the chitosan to the skin is sufficiently weakened to allow removal without disruption to the healing wound or the skin surface.

The hemostatic dressing assembly 400 may be held, bound, attached, or adhered to the compressive band support surface (including the support surfaces of any of the compression plate, the strap, and the compressive cushion component) using various methods. In a preferred embodiment, the hemostatic dressing 90 is connected to compressive band support surfaces 34 and 77 via a pressure sensitive adhesive (PSA) film layer 94. A preferred embodiment of detachment method of the tab film 98 from the compressive band support surfaces 34 and 77 is provided by use of a release surface coating (not shown) on the non-injury facing side of the tab film 98 surface. The release surface coating provides a reduced loading, clean release of PSA film layer from the tab film 98 surface when activated through the release tab 92, and provides for substantial absence of residual adhesive on the non-injury facing release film surface of the detached hemostatic dressing assembly 400. The release surface provides sufficient support of the hemostatic dressing assembly 400 to the compressive band 300 by the PSA film 94 during storage, and on initial application of the compressive band and hemostatic dressing system 200; however the release film 98 surface also allows for ease of removal of the hemostatic dressing 90 from the compressive band 300 by release tab 92 application. Release surfaces are typically achieved with thin coatings of low surface energy materials applied to a support film surface. Low surface energy materials include silicones, long alkyl chain branched polymers and fluorinated polymers. Alternate embodiments of release surface may be obtained without use of low surface energy thin coatings by use of a rough surface that reduces contact area of the PSA film layer 98 with the release surface. Alternate embodiments for release of the hemostatic dressing system 400 from the compressive band support surface may include controlled, or reduced surface area of contacting adhesive such as short lines of adhesive material or individual low surface area dots of hot melt or similar moderate strength adhesive. In a preferred embodiment, the hemostatic dressing 90 may be unbound, detached, unattached, released, or removed from the compressive band support surface on application of moderate load directed between normal to parallel of the adhesive surface(s). Once applied, the hemostatic dressing 90 may be unbound, unattached, or removed from the puncture injury by wetting with water or saline.

The compressive band and hemostatic dressing system 200 and method used to provide non-occlusive bleeding control, arterial injury treatment, and injury protection comfortably and reliably after a percutaneous vascular access procedure is preferably packaged and sealed within a dry, low moisture vapor transmissible (MVTR), heat sealable, foil or metalized paper pouch, or pre-formed foil or metalized container packaging system 170 (not depicted). A preferred embodiment of the invention provides a disposable packaging support insert 172 that is used to provide support and protection to the compressive band and hemostatic dressing system 200 of the invention within the packaging system 170. The packaging support insert 172 provides that the strap 70 and positioner assembly 10 are secured around its rigid or semi-rigid body to protect the hemostatic dressing system 400. In the case of compressive band and hemostatic dressing system 400 with a balloon component, the packaging support insert 172 includes a recess that secures and supports the pump. The compressive band and hemostatic dressing system 200 are secured to the packaging support insert 172 immediately before introduction into the packaging system 170. After sealing of the packaging system 170, the package, and its contents are sterilized by terminal gamma irradiation sterilization to at least sterility assurance level (SAL) 10⁻⁶. The sterilized and packaged system will have a shelf life when stored at or below ambient room temperature (≤26° C./79° F.) of at least 48 months.

The present invention may be held in place, or secured to a subject or a patient receiving treatment, using any of various mechanisms. For example, the present invention may be secured like a conventional watchband, via a ratcheting mechanism, by snaps, by hook and loop closure patches or tabs (e.g., Velcro®), etc. In a preferred embodiment, the present invention is secured to a subject or a patient receiving treatment like a watchband with hook and loop closure patches or tabs. In a preferred embodiment, the hinge mechanism 72 between the positioner assembly 10 and the compression plate 50 provides for initial application pressure by light to moderate hold pressure (applied between thumb and forefinger) from the caregiver. This pressure is maintained in the compressive band and hemostatic dressing system 400 on initial application and strap 70 securement to a patient's arm or leg. The securing mechanisms may be added to, connected to, attached, or integrally formed as part of the strap 70 and, optionally, the positioning assembly 10. The securing mechanisms are configured to be provided to, or removed from, a subject or treated patient together with the positioner assembly 10, compressive cushion component 30 (when used), compression plate 50, and strap 70 from an injury site.

As shown in FIGS. 11a, 11b, and 11c, a flexible hinge mechanism 72 between the positioner assembly 10 and the compression plate enables a simple initial application of pressure by a light to moderate pincer hold (applied between thumb and forefinger) from the caregiver that can be maintained in the compressive band and hemostatic dressing system 400 on initial application and strap 70 securement around a patient's arm or leg.

Preferred embodiments of the balloon component 32 are shown in FIGS. 5a, 5b, 5c and 5d. The balloon component is assembled of parts of an open balloon bladder body 36, an airline connector sleeve 38, an airline 110, an airline valve connector 152, and valve 150. The open balloon bladder body 36 is folded 180° on itself around the balloon bladder central fold line 44 such that all adjacent edges of the open balloon bladder body 36 overlay with each other to allow consistent, uniform and airtight joining of all free edges of the open balloon bladder body 36 against an adjacent free edge or a connection insert. Connector inserts may include, but not be limited to, tubes, tube connectors, filling ports, extruded lumens, airline connector sleeves 38, and connector films. Preferably the consistent and uniform airtight joining is provided by a welding technique such as external heat, induction, laser, solvent, ultrasonic, dielectric or radiofrequency welding. Preferably the welding together of adjacent edges of the open balloon bladder body creates a balloon bladder body first weld seam 46 between adjacent edges or connection insert. Preferably the width of balloon bladder body first weld seam 46 is at least 1 mm, more preferably the seam width is at least 2 mm, most preferably this balloon bladder body first weld seam 46 width is at least 3 mm. The completely closed, edge-welded balloon bladder body provides the balloon bladder 36.

Connection of the balloon bladder 36 to the airline 110 is by direct weld connection of the outer tube surface of the airline 110 or an airline connector sleeve 38 used as connection inserts during welding of the adjacent edges of the open balloon bladder body 36. Preferably, for welding attachment of a connector insert, such as an airline 110 or airline connector sleeve 38, there is need for increased weld seam width to provide for assurance of an airtight connection and resistance to pullout load. Preferably the width of the airline connector weld seam 48 in the axial direction of airline connector sleeve 38 insert is at least 3 mm width, more preferably it is at least 5 mm width, most preferably it is at least 7 mm width. In the case that the airline connector sleeve 38 insert is tube airline connector sleeve 38, then the width of the airline connector weld seam 48 at 90° transverse to the longitudinal axis of the tube is preferably half the outer diameter of the tube multiplied by 3.14159 with at least 1.5 mm extra seam width of balloon bladder body either side of the tube; more preferably it is half the outer diameter of the tube multiplied by 3.14159 with at least 2.5 mm extra seam width of balloon bladder body either side of the tube; and most preferably it is half the outer diameter of the tube multiplied by 3.14159 with at least 4 mm extra seam width of balloon bladder body either side of the tube. In the case that the airline connector sleeve 38 insert is a winged profile airline connector sleeve 38, then the width of the airline connector weld seam 48 at 90° transverse to the longitudinal axis of the airline connector sleeve 38 is preferably half the circumference of the winged profile airline connector sleeve 38 with at least 0.5 mm extra seam width of balloon bladder body either side of the connector sleeve; more preferably it is half the circumference of the winged profile airline connector sleeve 38 with at least 1.5 mm extra seam width of balloon bladder body either side of the connector sleeve; and most preferably it is half the circumference of the winged profile airline connector sleeve 38 with at least 2.5 mm extra seam width of balloon bladder body either side of the connector sleeve.

The airline connector sleeve 38 and the airline connector weld seam 48 extending from the balloon bladder 36 edge longitudinal with the axis of the airline connector sleeve 38 and at 90° to the longitudinal axis of the airline connector sleeve 38 provide for the airline connection balloon bladder neck 42.

Direct weld connection of the airline 110 to the balloon bladder 36 may not be possible due to welding material incompatibility between the material in the airline 110 and the material in the balloon bladder 36, or the size and compliance of the airline 110 cross-section. A preferred embodiment that addresses the problems of direct weld connection of the airline 110 to the balloon bladder 36 is attachment of the airline 110 to the airline connector sleeve 38 that is compatible with being welded to the balloon bladder 36. The airline connector sleeve 38 may be formed of an extruded profile such as a tube, or it may be molded, cast or machined. Preferred embodiments of the airline connector sleeve 38 are presented in FIGS. 5a, 5b, 5c and 5d. The winged profile airline connector sleeve 38 is preferably formed by a profile extrusion process, whereby the outer surface of the airline connector sleeve 38 has a winged profile, and the inner surface has a profile suitable for mating with the airline 110. The advantage of the winged profile airline connector sleeve 38 is that the integrity of welding of the profile to the balloon bladder 36 is more consistent than direct welding to a tube. Weld compression may be applied consistently and uniformly around the outside surface of the winged profile airline connector sleeve 38 to achieve consistent and integral sealing without adversely affecting the inside connector profile. In contrast, a tube shape may not be consistently and integrally welded at the weld seam edges of the tube, and also the internal tube profile may collapse under the increased weld compression needed to provide for weld integrity at the edges of the tube airline connector.

The inner surface of the airline connector sleeve 38 is shaped to closely mate with the outer surface of the airline 110 end that is fitted into the airline connector sleeve 38 to achieve an airtight and strong connection when finally attached. Final and permanent airline 110 end attachment inside the airline connector sleeve 38 may be provided by a welding technique such as external heat, induction, laser, solvent, ultrasonic, dielectric or radiofrequency welding. An alternative attachment of the airline 110 inside the airline connector sleeve may be provided by a glue technique that may include but is not limited to application of a cyanoacrylate or solvent glue to the external surface at the end of the airline 110 and the internal surface of the airline connector sleeve 38. A flaring or swaging process at the airline 110 end may be applied to provide for enhancement in the securing and airtight properties of the attachment of the airline 110 to the airline connector sleeve 38. This flaring or swaging process is performed by a flaring or swaging tool when the airline 110 end, to be mated, has been passed through the first end of the snugly fitting lumen of the airline connector sleeve 38 and through to the other end of the lumen, and is either seated outside the other end of the lumen of the airline connector sleeve 38 or is seated to align with the other end of the lumen of the airline connector sleeve 38. The former, with airline 110 end outside the other end of the airline sleeve connector, provides for enhanced integrity in a glued attachment by drawing the tightly fitting flared or swaged airline 110 end containing glue back into the lumen of the airline connector sleeve 38. The later, with airline 110 seated to align with the other end of the lumen of the airline connector sleeve 38 provides use of the flaring process to also be used in a welding manner to integrally attach and seal the airline 110 end with the other end of the airline connector sleeve 38. The final and permanent airline 110 end attachment inside the airline connector sleeve 38 may be provided by a welding technique such as external heat, induction, laser, solvent, ultrasonic, dielectric or radiofrequency welding.

A preferred embodiment with use of the tube airline connector sleeve 38 in the case of glue connection of the airline 110 to the tube airline connector sleeve 38 includes requirement for an excess of tube airline connector sleeve length protruding beyond the airline connection balloon bladder neck 42. Preferably the tube airline connector sleeve 38 attachment to the balloon bladder 36, should have at least 2 mm of tube airline connector sleeve 38 protruding from the edge of the balloon bladder neck 42 away from the neck weld seam. More preferably the tube airline connector sleeve 38 attachment to the balloon bladder 36, should have at least 4 mm of tube airline connector sleeve 38 protruding from the edge of the balloon bladder neck 42 away from the neck weld seam. Most preferably the tube airline connector sleeve 38 attachment to the balloon bladder 36, should have at least 7 mm of airline connector sleeve 38 protruding from the edge of the balloon bladder neck 42 away from the neck weld seam.

The airline 110 may be applied to the end of the airline connector sleeve 38 at the balloon bladder neck by insertion of the outer tube surface of one airline 110 end within the inner tube surface of the airline connector sleeve 38. Preferably the airline 110 end placed inside the airline connector sleeve 38 should fit snugly within the sleeve and should be able to be pushed into the sleeve to a depth of at least 2.5 mm. More preferably the airline 110 end placed inside the airline connector sleeve 38 should fit snugly within the sleeve and should be able to be pushed into the sleeve to a depth of at least 4 mm. Most preferably the airline 110 end placed inside the airline connector sleeve 38 should fit snugly within the sleeve and should be able to be pushed into the sleeve to a depth of at least 5.5 mm. Preferably the airline 110 end is permanently sealed inside the airline connector sleeve 38 to a depth of at least 2.5 mm to form a permanent airtight seal. More preferably the airline 110 end is permanently sealed inside the airline connector sleeve 38 to a depth of at least 4 mm to form a permanent airtight seal. Most preferably the airline 110 end is permanently sealed inside the airline connector sleeve 38 to a depth of at least 5.5 mm to form a permanent airtight seal.

The airline 110 is preferably connected to the valve 150 using an airline valve connector 152 such as a butterfly connector. A preferred method of connection of the airline 110 to the airline valve connector 152 is passing the airline 110 end fully through the snugly fitting lumen of the airline receiving end of the airline valve connector 152 and out the other end. Glue may be placed on the airline 110 end before withdrawing the end back into the airline valve connector 152 airline lumen to bond with the body of the airline valve connector 152 channel and to create a permanent airtight seal. The glue may include cyanoacrylate and solvent glues. Preferably a flaring or swaging process at the airline 110 end may be applied to provide for enhancement in the securing and airtight properties of the attachment of the airline 110 to the airline valve connector 152. This flaring or swaging process is performed by a flaring or swaging tool when the airline 110 end, to be mated, has been passed through the first end of the lumen of the airline valve connector 152 through to the other end of the lumen, and is either seated outside the other end of the lumen of the airline valve connector 152 or is seated to align with the other end of the lumen of the airline valve connector 152. The former, with airline 110 end outside the other end of the airline valve connector 152, provides for enhanced integrity in a glued attachment by drawing the tightly fitting flared or swaged airline 110 end containing glue back into the lumen of the airline valve connector 152. The latter, with airline 110 seated to align with the other end of the lumen of the airline valve connector 152 provides use of the flaring process to also be used in a welding manner to integrally attach and seal the airline 110 end with the other end of the airline valve connector 152. The final and permanent airline 110 end attachment inside the airline valve connector 152 may be provided by a welding technique such as external heat, induction, laser, solvent, ultrasonic, dielectric or radiofrequency welding.

The airline valve connector 152 with airline 110 attached is finally connected to the valve 150. The valve 150 and airline valve connector 152 have ends that are fashioned to mate to form a permanent airtight seal. A glue, such as a cyanoacrylate or a solvent glue, is used to ensure the airline valve connector 152 remains connected to the valve 150. The other end of the airline valve connector 152 is connected to the airline 110. The other end of the valve 150 is fashioned to provide an airtight connection to the syringe 132 to enable air to be added or withdrawn from the balloon component 32. The other end of the valve 150 also may be used to connect to a pressure gauge test system 190 to provide an airtight connection and that can be used to demonstrate consistent and reliable compression pressure delivery and maintenance in the balloon component 32 before it is accepted for attachment to the compressive band system 300.

A preferred embodiment of a manual pressure gauge test system 190 is provided in FIG. 5f. The preferred pressure gauge test system 190 is provided with a pressure dial gauge 192 with accuracy of at least ±5% able to read pressure of a range of 0 to 15 pounds per square inch. An example of the preferred dial gauge is Winters (www.winters.com) part number PEM136, series steel dual scale pressure gauge, 0-15 psi/kpa, 2″ dial display, ±3% accuracy and ¼″ NPT bottom mount. The dial gauge 192 is connected to a T-connector body 194 by a secure, airtight connection. The two arms of the T-connector body are connected to bidirectional, activated valves 196 through individual feed tubes 198 airtight connections. The activated valves 196 can be connected by airtight connections to syringe 132 or to valve 150. Typically, for valves 196, one valve is male and the other is female. The individual feed tubes 198 may be 2 cm to 12 cm lengths of Saint Gobain® C-Flex® tubing with internal diameter 3.2 mm and outer diameter 6.4 mm. The pressure gauge test system 190 may itself be pressurized by addition of air from connected syringe 132. Preferably, on removal of the syringe from the pressure gauge test system 190, the pressure gauge test system 190 may be validated for its airtight performance as demonstrated by raising pressure inside the system 190 to near 10 pounds per square inch and observing no more than 1 pound per square inch change over 3 minutes and testing by submersion in a water bath and observing absence of any bubbles appearing from the system (bubble test). More preferably, on removal of the syringe from the pressure gauge test system 190, the pressure gauge test system 190 may be validated for its airtight performance as demonstrated by raising pressure inside the system 190 to near 10 pounds per square inch and observing no more than 0.5 pound per square inch change over 3 minutes and testing by submersion in a water bath and observing absence of any bubbles appearing from the system (bubble test). Most preferably, on removal of the syringe 132 from the pressure gauge test system 190, the pressure gauge test system 190 may be validated for its airtight performance as demonstrated by raising pressure inside the system 190 to near 10 pounds per square inch and observing no more than 0.25 pound per square inch change over 3 minutes and testing by submersion in a water bath and observing absence of any bubbles appearing from the system (bubble test). A subsequently validated pressure gauge test system 190 may be used to conveniently test seal integrity of a balloon system without requirement to submerge the balloon system to observe for leaks. Such submersion testing is highly undesirable in routine acceptance testing of medical device balloons since the water submersion may damage the valve systems, or contaminate the balloon surfaces. Also, a wet balloon will require a period of drying.

An increase in width of the balloon bladder body first weld seam 46 at the opposite end of the balloon bladder 36 relative to the balloon bladder end with the balloon bladder central fold line 44 provides a second weld seam securement region of the balloon component 32 that can be attached to the compressive band system 300 under the compression plate 50 without risk of compromising an accepted balloon component 32 in the process of attachment. The width of the balloon body first weld seam 46 at the opposite end of the balloon bladder 36 relative to the balloon bladder end with the central fold line 44 is preferably is at least 3 mm width, more preferably it is at least 4.5 mm width, and most preferably it is at least 6 mm width. The balloon bladder 36 to compression band second weld seam 40 securement is preferably provided on the compressive band system 300, under the compression plate 50, using the second weld seam 40 that is at least 1 mm in width and at least 1 mm clear of the inside edge of the balloon first weld seam 46. The balloon bladder second weld seam 40 securement is more preferably provided on the compressive band system 300 under the compression plate 50 using the second weld seam 40 that is at least 2 mm in width and at least 1.5 mm clear of the inside edge of the first balloon weld 46. The balloon bladder second weld seam 40 securement is most preferably attached to the compressive band system 300 under the compression plate 50 using the second weld seam that is at least 3 mm in width and at least 2 mm clear of the inside edge of the balloon first weld seam 46. The balloon bladder second weld seam 40 securement is preferably under the compression plate, attached to the strap pocket 76 on the edge of the compression plate 50 opposite the edge of the compression plate 50 that is attached to the positioner assembly 10. The present invention may or may not include markings to facilitate correct positioning of the hemostatic patch on a puncture injury.

### Example 1. HemCon Bandage Treatment of Arterial Injury in a Femoral Access Sheep Injury Model

Background. The femoral artery injury model in sheep was developed by the Oregon Health & Science University Dotter Institute to investigate hemostatic efficacy of percutaneous arterial closure devices (PACD's) compared to standard of care manual compression (Ni et al. 2009). The Dotter Institute sheep model provides a significant advantage over previous preclinical models of investigation of hemostatic efficacy of PACD's in that the Dotter model includes fluoroscopic angiographic imaging of the arterial injury site with ability to directly visualize arterial injury presence or absence of extravasation (bleeding). The 8-French sheath injury size used in this study creates a level of anti-coagulated (heparinized) bleeding that provides a challenging model for standard of care manual compression and is therefore a good model to investigate PACD arterial bleeding control treatment. The Dotter institute had previously investigated a smaller 6-French size sheath injury and found it unsatisfactory for challenging a treatment arm (Ni et al. 2009). Ni et al. reported use of a different chitosan dressing Clo-Sur PAD (Scion Biomedical) in this model development (Ni et al. 2009) with the 8-French (F) sheath introducer with success rate at 5 minutes application less than 50%. Although the study set results presented herein are substantially unpublished, the Dotter Institute has previously reported on hemostatic performance of the HemCon Bandage in percutaneous vascular closure in the sheep model (Kim et al. 2009 & Kranokpiraksa et al. 2010).

Materials. HemCon Bandages, 5 F angiography catheter, 8 F introducer sheath, 0.035 guide wire, ACT machine (HEMACRON 801).

Animal Testing. All testing was carried out in healthy sheep between 100 to 140 lbs. All experiments are performed in accordance with the 1996 National Research Council, “Guide for the Care and Use of Laboratory Animals.” The sheep underwent one acute procedure at the Dotter research lab. Prior to delivery in the morning, the animals were fasted overnight with water ad libitium.

The sheep (N=19) were tranquilized with ketamine (2.0 mg/lb (100 mg/ml) and midazolam (0.05 mg/lb (5 mg/ml), IV), and 4 to 5 mL of atropine to control salivation, then intubated and maintained at 1.5 to 2% isoflurane breathing spontaneously or on a mechanical ventilator. The animals' vital signs and depth of anesthesia were monitored by expired CO₂ and pulse oximetry.

The inguinal and neck areas of the sheep were shaved and cleaned. A 5 F sheath and 5 Fr catheter were placed in the carotid artery for angiography. The animals were heparinized with 100 units/kg administered intravenously (IV). Activated clotting times (ACT) were monitored at baseline, prior to applications of treatment or control, and at the end of the procedure. The femoral artery was punctured percutaneously with an 18 gauge needle guided by an angiographic roadmap and an 8 F sheath was placed in the femoral artery.

One side served as the control, receiving standard manual pressure applied over the puncture site while the contra-lateral side will receive treatment with the HemCon Bandage and application of pressure.

The application of pressure for the control or treatment (HemCon Bandage) proceeded as follows:
1. Remove sheath following 5 minutes of placement in the femoral artery.
2. Apply pressure proximal to the puncture site area (point on the skin at which the sheath is inserted and removed).
3. Dry the treatment area.
4. Allow slight bleed (approximately the size of a nickel) for the HemCon treatment side only and then apply the HemCon bandage over treatment area.
5. Apply pressure to the puncture site area for 5 min. and then slowly release proximal pressure.
6. Perform angiography to confirm whether hemostasis has been achieved and check for bleeding at puncture site.
7. If there is evidence of bleeding, either visual or angiographic, continue applying additional pressure and reevaluate.
8. Reapplication of pressure is continued until hemostasis is achieved.
9. Perform follow-up angiogram within at least 30 min. after sheath removal.

Success criteria for achieving hemostasis was angiographic confirmation of lack of extravasation. Data was collected on number of applications of pressure to achieve hemostasis, time to achieve hemostasis confirmed by angiography and visual inspection, angiographic rank of extent of bleeding and evidence of hematoma.

The animals were euthanized while still under general anesthesia with 5 mL of Euthasol delivered intravenously.

A test piece of HemCon Bandage dressing was to be excluded if the packaging was breached or if the dressing appeared damaged. Single-tailed, paired t-tests and/or Wilcoxon Rank tests were performed to determine if there is a statistically significant difference (p≤0.05) between the control and treatment.

Results. The results of preclinical hemostatic testing of the chitosan HemCon Bandage dressing in the sheep femoral injury model are tabulated in FIG. 12. Mean times (±standard deviation) to visual cutaneous and angiographic vascular hemostasis for HemCon Bandage treatment and manual compression control are shown below:

Hemostasis time (min.)

| | Cutaneous | Angiographic vascular |
|---|---|---|
| Treatment | 5.4 (±1.3) | 6.8 (±3.5) |
| Control | 6.4 (±1.7) | 10.1 (±3.8) |

The single-tailed, paired t-tests demonstrated p-values of difference between treatment and control of 0.13 and 0.006 for cutaneous and vascular time to hemostasis respectively. The incidence of hematoma between treatment and control is highly significant (Wilcoxon p-value=0.002) with 3/19 (16%) of HemCon Bandage chitosan treated injuries showing slight hematoma while 14/19 (73%) of control treated injuries demonstrated hematoma 7/19 (37%) slight, 5/19 (26%) moderate and 2/19 (11%) significant.

In the treatment arm, the close to insignificant (p-value=0.053) 1.4 minutes mean hemostasis time difference between cutaneous hemostasis (virtually immediate) and angiographic vascular hemostasis contrasts with the significant difference (p-value=0.0004) of 3.7 minutes between control cutaneous hemostasis and angiographic hemostasis. The significant longer mean bleeding time of 3.7 minutes is responsible for the significant level of hematoma in in the control arm. Given absence of any added pharmaceutical active ingredients in the HemCon Bandage, the comparatively rapid stanching of bleeding at the vascular access site by a mucoadhesion mechanism of action in this challenging anti-coagulated model, following application of the HemCon Bandage at the cutaneous access site some 2 to 3 mm distant from the artery, is both remarkable and unexpected. Clinical study results (Arbel et al. 2010) are consistent with this unexpected ability of the HemCon chitosan dressing to treat an injured artery following a percutaneous procedure by application of a HemCon chitosan dressing at the cutaneous injury site.

### Example 2. Consistency and Reliability of Balloon Compression System of the Invention

Background Consistent and reliable compression pressure delivery and maintenance is a critical design consideration in a vascular compression device. Inability to deliver and maintain pressure, and, in particular, gross pressure system delivery failure in hemostatic vascular compression devices could result in harm to a patient.

The balloon systems employed to deliver compression pressure in many vascular devices are susceptible to inadequacies and failures. Medical device manufactures are required to report adverse events to the US Food and Drug Administration (FDA) Center for Devices and Radiological Health (CDRH). MAUDE (Manufacturer and User Facility Device Experience) is a database of adverse events reported for medical devices. The online interface searches the CDRH database for information on medical devices that may have malfunctioned or caused/contributed to a death or serious injury. An online search of the MAUDE database was conducted in May 30, 2015 for US FDA for reported adverse events in cleared hemostatic vascular compression devices utilizing balloon compression with search term names TRBand™, TR Band®, RADAR™ Vascular Compression, Airband™, Vasc Band™ over the period Jan. 1, 2000-May 30, 2015.

Product code “DXC” AND Manufacture “Terumo” resulted in 78 hits for “TR BAND®” between April 2010 and April 2015. There were 59 reported failures associated with balloon integrity that resulted in incidences of hematomas and failures to treat. Two hematomas were reported without suggestion of a balloon failure incident. There was an additional hematoma reported that was in conjunction with a suspected compartment syndrome. Three cases of “swelling” associated directly with use of the TR Band® and inflation with 14 mls of air were reported. There was one reported unintentional removal of the band during hemostatic use by unknown means. Four cases of failure to treat hemostasis were reported which may have been associated with balloon integrity but which was not discussed. There was one report of hand numbness following TR Band® use. There was a reported failure of the Terumo syringe filling-tip. There were reports of receipt of damaged TR Bands®, primarily the hook and loop fastener adhering to the balloon and damaging the balloon when removed as damaged in shipment. There was one report of the balloon filling tube as damaged. There were 16 reported Vasc Band™ failures between October 2013 and April 2015. There were 11 hematomas associated directly with Vasc Band™ use. There was only one reported balloon integrity failure of the Vasc Band™. There were two reported cases of unintended removal of the Vasc Band™ during use. There were two reported incidents of Vasc Band™ distal swelling/redness associated directly with compression band use.

Test 1. Cycle Balloon Loading with Discrete Volume Increments of Fill on 2 inch Diameter Rigid Pipe Fixture

Materials. Non-sterile, prototype compressive band with balloon component of the invention (A) with a balloon bladder inner seam edge dimensions 3.0 cm×4.0 cm and including hemostatic dressing assembly.

Syringe

Mylar supported, 9.53 mm diameter FlexiForce® B201-L Sensor (low gain setting) (www.TEKSCAN.com)

Flexiforce® ELF™ data acquisition handle (system interface to connect sensor to PC)

Personal computer running Tekscan® ELF software

Rubber disk (9.5 mm diameter×3.3 mm thickness Shore 50A rubber disk)

500.0 g calibration load standard

1000.0 g calibration load standard

Clear 3M single-sided, 19 mm wide, 2 mil adhesive tape

10 cm length of 2″ diameter polyvinylchloride rigid pipe

Timer to 120 minutes

Method. The rubber disk was placed centrally over 9.53 mm diameter Fexiforce® B201-L sensor, and both sensor and rubber disk were taped in place on a rigid flat bench surface. The sensor was connected to the personal computer running the Tekscan® ELF software. The load force was calibrated to within ±5% using the 500.0 g and 1000.0 g calibration standard weights. The rubber disk and FlexiForce® sensor were subsequently removed from the rigid flat bench surface, and the FlexiForce® sensor was taped in place on the external surface of the rigid pipe. The prototype (A) compressive band and balloon used in the experiment had previously been conditioned substantially to remove balloon expansion under load by inflating and deflating through at least 10 cycles of filling with 12 milliliters (mL) of air under load on the rigid fixture. Typically in light pressure manual control of arterial bleeding using a HemCon chitosan dressing, 300 g to 500 g of digital load is applied immediately onto the dressing over the wound.

Procedure Cycle 1. The test prototype compressive band A was attached, using minimal positive loading application pressure, using minimal positive loading application pressure, to the rigid pipe with the balloon bladder of the system centrally positioned over the load cell sensor with strap attachment. The initial load reading from strap attachment of the empty balloon bladder on the load cell was recorded. The syringe was then loaded with 8 milliliters (mL) of air under normal ambient room temperature conditions (between about 20 to 26° C./68 to 79° F.) and attached to the valve. The 8 mL of air was transferred to the balloon attached to the rigid fixture and the load was recorded within 1 minute of loading and after 30 minutes. The syringe was removed. At 30 minutes, the syringe was reconnected to the valve and 1 mL of air was removed from the system and the syringe was disconnected. The balloon bladder load with 7 mL balloon volume was recorded within 1 minute of air removal and at 15 minutes after air removal. At 45 minutes, the syringe was reconnected to the valve and another 1 mL of air was removed from the system and the syringe was disconnected. The balloon bladder load at 6 mL balloon volume was recorded within 1 minute of air removal and at 15 minutes after air removal. At the conclusion of this step, the balloon bladder was emptied of air, a final load reading was noted and the compressive band system (without a hemostatic dressing assembly) was removed from the fixture. The above procedure cycle was repeated to test for consistency on addition of air to the balloon. Procedure Cycle 2. An additional procedure cycle that included an addition of 12 mL of air in the balloon was performed as follows. The test prototype compressive band (A) was attached, using minimal positive loading application pressure, using minimal positive loading application pressure, to the rigid pipe with the balloon bladder of the system centrally positioned over the load cell with strap attachment. The initial load reading from strap attachment of the empty balloon bladder on the load cell was recorded. The syringe was then loaded with 8 mL of air under normal ambient room temperature conditions (between about 20 to 26° C./68 to 79° F.) and attached to the valve. The 8 mL of air was transferred to the balloon attached to the rigid fixture and the load was recorded within 1 minute of loading and after 30 minutes. The syringe was removed. At 30 minutes, the syringe was reconnected to the valve and 4 mL of air was added to the system and the syringe was disconnected. The balloon bladder load with 12 mL of balloon volume was recorded within 1 minute of air removal and at 30 minutes after air addition to 12 mL. The balloon bladder was emptied of air, a final load reading was noted, and the compressive band system (without a hemostatic dressing assembly) was removed from the fixture. The above procedure cycle was repeated (with a 15 minute hold at 12 mL balloon bladder volume) to test for load reading consistency on addition of air to the balloon.

Results and Discussion. On zero (0) mL of balloon volume fill, the initial load readings from strap attachment of the empty compression balloon on the load cell were found to be 129 (±27) g over the 4 initial strap attachments. At conclusion of the loading cycle with removal of all air from the balloon, the load registered near 0 gram indicating a relaxation near −130 g in the strap fixture load following loadings to 600 g or above. This is likely a small relaxation under tension in positioning of the hook and loop fasteners on the strap when placed under load around the rigid pipe fixture.

Average load and consistency in loading (error bars) following discrete volume balloon addition and volume removal are plotted in FIG. 13 for initial load (pre-relaxation) and load after 15 to 30 minutes relaxation (post-relaxation). The linear correlation coefficients of pre-relaxation and post-relaxation loads plotted against balloon volume fill are R²=0.99. The linear gradients for pre and post relaxation are 73 and 77 g/ml respectively.

In the case of initial loadings with 12 ml and 8 ml of air, the relaxation of the band load over 30 minutes averaged −110 (±30) g and −48 (±40) g, respectively. There was no significant relaxation (+6 g) over 15 minutes in the 7 mL volume fill after loading at 8 mL initially for 30 minutes. These results are consistent with the proposed hook and loop band relaxation effect.

The results of this reliability and consistency testing of the compressive band and balloon on a rigid fixture demonstrate that the balloon and band system (A) substantially meets requirements for extended and consistent pressure application in the target range of 300 g to 500 g of directed load. Also the results demonstrate ability to increase or reduce applied load by balloon inflation or deflation respectively.

Conversion of the load to internal balloon compression may be estimated from the surface area of the rubber disk (0.713 cm²)

| Load | Pressure |
|---|---|
| (grams) | (pounds per square inch (psi)) |
| 909 | 18.1 |
| 621 | 12.4 |
| 507 | 10.1 |
| 374 | 7.46 |

Test 2. Test Method Sensitivity and Balloon Reliability Testing by Unloaded Balloon Inflation to 7 psi and Holding for 10 minutes and 120 minutes.

Materials. Non-sterile, sacrificial prototype compressive band (N=1) with balloon component of the prototype (A) with balloon bladder inner seam edge dimensions 3.0 cm×4.0 cm.

Non-sterile, prototype compressive bands (N=302) from engineering prototype Lot run with balloon component of the invention (A) with balloon bladder inner seam edge dimensions 3.0 cm×4.0 cm.

Pressure gauge test system.

Timer to 120 minutes.

Submersion water bath for bubble test.

Uson® standard 12.5 micron diameter laser bored hole patch.

Syringe

### Methods

The sacrificial prototype balloon component from compressive band and hemostatic dressing system was inflated with air to 10 pounds per square inch as verified by the pressure gauge test system and then submerged in a water bath to demonstrate absence of any air bubble leaks from the balloon component. A small 2 mm diameter hole was punched through a single wall in a middle region of the sacrificial prototype balloon bladder and a Uson® disk with standard 12.5 micron diameter hole was placed and over the 2 mm diameter punched hole with integral sealing of the patch to the original hole. The prototype balloon component was connected through valve to the pressure gauge test system. The syringe was then attached to the free end of the pressure gauge test system and enough air was slowly delivered to cause a pressure rise to 5 pounds per square inch (psi). Decay of the pressure from 5 psi through loss of air from the controlled 12.5 micron hole was followed in 5 separate tests on the same device. Water bath submersion was used to verify that air bubble leakage was exclusively through the 12.5 micron hole.

Having demonstrated sensitivity and reliability of the pressure gauge test system, the pressure gauge test system was used to test 302×balloon components of the invention from 302×prototype compressive bands from an engineering prototype Lot run. The testing protocol used (at ambient room temperature between about 20 to 26° C./68 to 79° F.) was as follows:
1. Using a syringe connected to a valve of pressure gauge test system, which remaining valve is connected to the valve of balloon component, and deliver sufficient air (generally near 10 ml) to raise balloon pressure to 7.0 psi.
2. Disconnect syringe.
3. After 1 minute system relaxation, reconnect syringe and adjust pressure back to 7.0 psi by addition or removal of air (generally addition of air since new balloons expand slightly on initial loading).
4. Disconnect syringe.
5. Allow 10 minutes test time after the pressure adjustment to 7.0 psi
6. The preferred acceptance of a balloon component is less than 1.0 psi pressure loss in 10 minutes after inflating balloon component to 7.0 psi.
7. Failed balloon components are tested in the water bath bubble submersion test to identify location of failure.

Having demonstrated 99% balloon component success in the 10 minute trial, 9 of the successful balloons were randomly selected for 120 minute testing. The test protocol is detailed below:
1. Using syringe, connected to the valve of pressure gauge test system, which remaining valve is connected to the valve of the balloon component, and deliver sufficient air (generally near 10 ml) to raise balloon pressure to 7.0 psi.
2. Disconnect syringe.
3. Record pressure (psi) at time=0, 5, 10, 20, 30, 60, 90 and 120 minutes.

Results. In the 12.5 micron diameter hole size pressure drop validation test (3.0×4.0 cm edge seam balloon component), the pressure loss at 3 minutes after filling to 5 psi was found as shown below:

| | Pressure drop (psi) & |
|---|---|
| | % change (bracketed) |
| Test Number | at 3 minutes |
| 1 | −1.8 (−36%) |
| 2 | −1.6 (−32%) |
| 3 | −1.7 (−34%) |
| 4 | −1.6 (−32%) |
| 5 | −1.5 (−30%) |

The outcome of pressure testing the 302×development lot balloon components 32 in the 10 minute hold was 298/302 successful and 4 unsuccessful. The results of the study are tabulated below:

| Outcome | Number | Observations |
|---|---|---|
| Success | 298 | Minimum and maximum pressure |
| | | losses were 0.25 psi & 0.75 |
| | | respectively. |
| Failure | 3 | All demonstrated close to 2 psi |
| | | pressure loss, welds were all |
| | | integral, sealing failures were at |
| | | glue join of airline end to airline |
| | | connector sleeve. All airlines |
| | | were originally connected by |
| | | placement of glue on a non- |
| | | flanged airline end outside the |
| | | airline connector sleeve before |
| | | placement inside the connector |
| | | sleeve to achieve bonding. All |
| | | airlines were originally connected |
| | | by placement of glue on a non- |
| | | flanged airline end outside the |
| | | airline connector sleeve before |
| | | placement inside the connector |
| | | sleeve to achieve bonding. |
| Failure | 1 | There was one 5 psi pressure loss |
| | | with sealing failure at airline |
| | | connector sleeve weld to balloon |
| | | bladder neck. |

The results of pressure testing the randomly selected 9 successful development lot balloon components over 120 minutes are tabulated in FIG. 14. The lowest 120 minutes pressure (band 4) was 4.7 psi with loss of 2.3 psi (−32%), which is within acceptance of the allowed negative 35% loss at 120 minutes. The next lowest 120 minutes pressure was 4.9 psi with a negative 30% loss. The remaining seven 120 minutes pressures of 5.4, 5.6, 5.6, 5.2, 5.4 and 5.6 psi for bands 1, 2, 3, 5, 6, 7 and 8 respectively have an average loss of negative 21.6±2.3%.

The overall average loss in pressure of near −1.67 psi (−24%) from the original 7.0 psi over two hours is substantially relaxation and yield in the polyvinylchloride film used to form the balloon bladder body. This is evident in that close to 50% of the change occurs within the first 10 minutes. If desired, this relaxation in the balloon body could be reduced or eliminated by pre-biaxial orientation of the balloon body material or use of a material with reinforcement or higher crystallinity or any combination of these approaches.

### Example 3. Ease of Application and Comfort of the Compression Band and Hemostatic Dressing System

Background. This study investigated comfort, ease of correct application placement, and load change of the compressive band system around non-injured, volunteer subject wrists for a period of 120 minutes after initial loading to a target load of 700 g.

A significant advantage of the hemostatic efficacy of the compressive band and hemostatic dressing system of the invention over pressure only compressive band systems is that, immediate bleeding control is achieved with as little as 300 to 500 g of pressure placed locally over the preferred hemostatic dressing embodiment comprising a mucoadhesive, sealing chitosan dressing. This immediate bleeding control with moderate to low local load application has been demonstrated manually in studies by HemCon in application of its HemCon Bandage chitosan dressing over 4 mm diameter perforation injuries in swine aortas as well as in smaller arterial injuries.

Significantly greater load (at least up to 30% more) is required in the case of a pressure only device to control arterial bleeding since such devices exclusively rely on partial to complete arterial occlusion to achieve hemostasis. Pressure only devices also require extended time of load application to enable device removal without risk of re-bleeding. The higher load required in pressure only hemostatic devices not only provides for less comfortable applications with extended time to hemostasis, but also subjects the patient to significantly higher risk of adverse events including neuropathy and radial artery occlusion.

Materials. Non-sterile prototype compressive band with balloon component of the invention (A) with balloon bladder inner seam edge dimensions 3.0 cm×4.0 cm and including hemostatic dressing assembly.

Syringe

Mylar supported, 9.53 mm diameter FlexiForce® B201-L Sensor (low gain setting) (www.TEKSCAN.com)

Flexiforce® ELF™ data acquisition handle (system interface to connect sensor to PC)

Personal computer running Tekscan® ELF software

Rubber disk (9.5 mm diameter×3.3 mm thickness Shore 50A rubber disk)

500.0 g calibration load standard

1000.0 g calibration load standard

Clear 3M single-sided, 19 mm wide, 2 mil adhesive tape

Timer to 120 minutes

Methods. Five volunteer subjects with different wrist sizes wore the compressive band system for 120 minutes with monitoring of load by pre-attachment of a FlexiForce® sensor over the radial artery on the wrist at 30, 60, 90 and 120 minutes. Local load over the wrist was applied through the balloon component by balloon inflation with 6 to 7 ml of air from syringe. Use of the band positioner assembly and the flexible hinge mechanism provided for reproducible and correct compressive band system placement. Application loads used were less than those that could cause arterial occlusion. However, the load applied was more than sufficient to control arterial bleeding in conjunction with a mucoadhesive hemostatic HemCon Bandage. The following protocol was used:
1. Wrist circumference was measured
2. The Flexiforce® sensor was calibrated on a hard, flat surface using 500.0 g and 1000.0 g calibration weights
3. The Flexiforce® sensor and centrally placed top rubber disk were taped in place over the radial artery on the right hand with the Flexiforce® mylar connection in alignment with the arm and extending distally away from the arm. When not connected to the computer running Tekscan® ELF software, the mylar FlexiForce® sensor connector was folded back on the arm.
4. The prototype compressive band with balloon component of the invention (1a) was placed around the wrist and secured according to its instructions for use.
5. The syringe was used to provide 6 to 7 mL of air inflation to the balloon component by attachment to and removal from valve.
6. Load on the FlexiForce® sensor was recorded immediately on addition of the air.
7. The FlexiForce® sensor was disconnected from the computer and the subjects were allowed to return to routine tasks between data collection periods with the compressive band with inflated balloon component attached around their wrists.
8. At time points of 30, 60, 90 and 120 minutes, the subjects returned to the test location for around 5 minutes. During this time, the FlexiForce® sensor was reconnected to the computer running Tekscan® ELF software and a load reading was recorded.
9. At the conclusion of the 120 minute testing period and after the last reading, the compressive band was removed from the subject's wrist and the subjects provided their comfort rank in wearing the band (see rank scale chart below). Finally, the FlexiForce® sensor load was tested with 500.0 g and 1000.0 g calibration weights to ensure that load measurement had remained within the original calibration settings.

Wearer Comfort Rank Scale

| RANK | DESCRIPTION |
|---|---|
| 1 | Mild pressure to wrist: comfortable at short and long times |
| 2 | Moderate pressure to wrist: comfortable at short and long times |
| 3 | Moderate pressure to wrist: comfortable at short (<5 mins) but |
| | less comfortable at longer times |
| 4 | High pressure: uncomfortable at all times |

Results. The results of comfort and load change of the compressive band system around non-injured, volunteer subject wrists for a period of 120 minutes after initial loading to a target load of 700 g are presented in the Table in FIG. 15.

Subject wrist load is plotted against time in FIG. 16. Load calibration before and after the study demonstrated that loads recorded during the study were valid. In FIG. 16, it is observed that initial load relaxes from 740 to 900 g to near 600 g within 20 minutes and stabilizes afterwards to between 500 g and 600 g load until 120 minutes when the band was removed.

The single applicator of the band during the study reported ease of correct application placement. Two subjects reported mild pressure to wrist and comfort at all times and three subjects reported moderate pressure on wrist and comfort at all times.

### Example 4. Antibacterial Barrier Testing and Stability of Antibacterial Properties

Objective. Wound contamination, and the infection resulting from wound contamination is a serious global problem. A hemostatic wound dressing with shelf life of four (4) years or more, that not only controls bleeding, but also provides >log 4 reduction of clinically relevant bacteria within 24 hours at the site of injury is highly desirable. The HemCon Bandage is known to provide bleeding control for at least four years when packaged and sterilized by gamma irradiation to sterility assurance level (SAL) 10⁻⁶ in an airtight foil package. To date there has been no similar demonstration of prolonged antibacterial activity stability in a chitosan dressing. Although wound healing properties are reported for chitosan, there are no studies demonstrating prolonged stability of wound healing properties of chitosan acid dressings.

Enhanced wound healing properties of chitosan acid salts are well demonstrated (Prudden et al. 1970, Stone et al. 2000, Kordestani et al. 2008, Baxter et al. 2012, Farrugia et al. 2014, Jung et al. 2013). The HemCon Bandage U.S. patent application Ser. Nos. 10/743,052 and 11/020,365 describe use of a HemCon Bandage as a protective antibacterial covering. In these patent applications, antibacterial testing of <1 year old, sterilized and packaged products is described. Testing was performed against freeze-phase-separated, freeze-dried, chitosan acetate, interconnected porous dressings. An in vitro colony counting, plating method, USP 27<51> was used against Staphylococcus aureus., Pseudomonas aeruginosa and Escheria coli demonstrating substantial Log-reduction (>Log 3 reduction) in viable bacteria colonies. A contaminated mouse injury model was used to investigate antibacterial activity against Pseudomonas aeruginosa, and Proteus mirabilis bacteria with controls of alginate dressing and standard of care silver sulfadiazine. Animal survival in full thickness incision dermal injury group treated by the chitosan acetate dressing was significantly greater than in animal groups treated with alginate or with silver sulfadiazine. The bacteria used in the in vivo study were stably transduced with the entire bacterial lux operon to allow in vivo bioluminescence imaging of bacterial viability. The significant loss in bacterial bioluminescence on exposure to chitosan acetate demonstrated the mechanism of antibacterial activity in chitosan acetate is bactericidal.

This study investigates the antibacterial activity of dry, chitosan acetate HemCon Bandage stored for more than four years. The antibacterial testing is against ten clinically relevant bacteria that have been shown to be involved in serious wound contamination and infection.

Materials. HemCon Bandage 4″×4″ Lot #10-101-018 was used for testing. Dressings were sterilized on Oct. 18, 2010 and were assigned an expiration date of Oct. 31, 2014.

| Test Organisms | Sources |
|---|---|
| Acinetobacter baumannii | ATCC 15308 |
| Enterococcus faecalis (VRE) | ATCC 700802 |
| Enterococcus faecalis (VRE) | ATCC 51299 |
| Moraxella catarrhalis | ATCC 8193 |
| Shigella species | ATCC 11126 |
| Staphylococcus aureus (MRSA) | ATCC 33591 |
| Staphylococcus aureus (MRSA) | ATCC BAA-1556 |
| Staphylococcus epidermis | ATCC 12228 |
| Streptococcus pneumonia | ATCC 10015 |
| Streptococcus pyogenes | ATCC 19615 |

Methods. The antibacterial barrier testing was performed under FDA, 21 CFR Part 58, “Good Laboratory Practice for Non-Clinical Laboratory Studies.” The standard method American Association of Textile Chemists and Colorists (AATCC), 2015 current version Test Method 100 “Assessment of Antibacterial Finishes on Textile Materials was used to provide a quantitative procedure for the evaluation of the degree of antibacterial activity. The standard USP chapter <1227> “Validation of Microbial Recovery” was used to demonstrate that a neutralization method was effective in inhibiting the antimicrobial properties of the product without impairing the recovery of viable micro-organisms. Testing was performed in duplicate. Acceptance criteria for antimicrobial reduction was a minimum 4-log reduction in test organism at 24 hour contact time as compared to initial contact time. Testing time of test Lot to demonstrate 48 month retention of bactericidal antibacterial activity was at 48 months or after 48 months of the sterilization date of the test article. Product was stored at ≤27° C. during the storage period.

Results. The results for the antibacterial testing are summarized below. The antibacterial testing of dressings stored for more than 48 months before testing met the acceptance criteria of minimum 4-log reduction in test organism at 24 hour contact time as compared to initial contact time.

| | | Log | % |
|---|---|---|---|
| | | Reduc- | Reduc- |
| Test Organisms | Sources | tion | tion |
| Acinetobacter baumannii | ATCC 15308 | >4.4 | >99.99% |
| Enterococcus faecalis (VRE) | ATCC 700802 | >4.7 | >99.99% |
| Enterococcus faecalis (VRE) | ATCC 51299 | >4.8 | >99.99% |
| Moraxella catarrhalis | ATCC 8193 | >4.0 | >99.99% |
| Shigella species | ATCC 11126 | >4.0 | >99.99% |
| Staphylococcus aureus (MRSA) | ATCC 33591 | >4.9 | >99.99% |
| Staphylococcus aureus (MRSA) | ATCC BAA-1556 | >4.2 | >99.99% |
| Staphylococcus epidermis | ATCC 12228 | >4.3 | >99.99% |
| Streptococcus pneumonia | ATCC 10015 | 4.2 | 99.99% |
| Streptococcus pyogenes | ATCC 19615 | >4.5 | >99.99% |

Conclusions. The present invention advantageously provides a stable system, that it is simple and easy to apply, provides a reliable and comfortable hemostatic system, and permits light to moderate pressure application such that it helps avoid incidence of adverse events such as neuropathy and arterial occlusion, and provides 24 hr antibacterial protection and wound treatment, thus reducing or removing the opportunity for expensive and problematic infections and rebleeding. System and method embodiments of invention address shortcomings of currently available commercial compression bands. Packaging, balloon embodiment and acceptance testing provide for very low probability («1×10⁴) of balloon failure on device.

1. A compressive band and hemostatic dressing device comprising:
a positioner assembly;
a strap comprising a flexible hinge assembly;
a compression plate; and
a releasably bound hemostatic dressing.

2. The device of claim 1, wherein the strap comprises a flexible hinge assembly that provides independent movement of the positioner assembly and the releasably bound hemostatic dressing.

3. The device of claim 1, further comprising a compressive cushion component on one of the compression plate or strap, and the hemostatic dressing is provided on the compressive cushion component.

4. The device of claim 2, wherein the hemostatic dressing comprises chitosan.

5. The device of claim 1, wherein the compression plate is substantially flat and is located in a pocket provided within the strap.

6. The device of claim 1, wherein the device is non-occlusive.

7. A compressive band and hemostatic dressing device comprising:
a strap comprising a flexible hinge assembly, a compression plate, a compressive cushion component, and a hemostatic dressing assembly;
a positioner assembly movably connected to a strap by the flexible hinge assembly; and
a hemostatic dressing releasably bound, attached, or connected to the compressive cushion component.

8. The device of claim 7, wherein the compression plate is rigid and substantially flat.

9. The device of claim 8, wherein the compressive cushion component is located entirely under the compression plate and strap with a flexible hinge assembly.

10. The device of claim 7, wherein the positioner assembly is flexible or semi-rigid with a fixed curve profile.

11. The device of claim 7, wherein the hemostatic dressing comprises chitosan.

12. The device of claim 11, wherein the hemostatic dressing is a phase-separated, freeze-dried chitosan acid dressing, including an acid salt comprising one of acetic, lactic, glycolic or citric acid.

13. The device of claim 7, wherein the compression plate is located in a pocket provided within the strap.

14. The device of claim 7, wherein the compression plate is located on a surface of the strap.

15. The device of claim 7, wherein the compressive cushion component is attached to one of the compression plate and the strap.

16. The device of claim 15, wherein the compressive cushion component is attached along only one edge to a surface that is off-center relative to the compression plate.

17. The device of claim 7, wherein the device is non-occlusive and hemostatic dressing assembly is attached to one of the compression plate, the strap, and the compressive cushion component.

18. A method of using the compressive band and hemostatic dressing device of claim 1, wherein the device comprises a compressive cushion component, including the step of leaving the hemostatic dressing in place after hemostasis is achieved and the positioner assembly, strap with a flexible hinge assembly, compression plate, and compressive cushion component, are removed from a puncture site.

19. The method of claim 18, further including the steps of placing the device with the aid of the strap flexible hinge assembly to independently move the positioner assembly and the hemostatic dressing, applying compressive force, and avoiding arterial occlusion.

20. The method of claim 18, further comprising locating the compressive cushion component entirely under the compression plate and strap so that it is not subject to direct compression by the positioner assembly.
